(19) United States
(12) Patent Application Publication
(10) Pub. No.: US 2013/0310457 A1
(43) Pub. Date: Nov. 21, 2013

(54) SOLID-IN-OIL DISPERSIONS

(71) Applicant: Niral Ramesh, Princeton Junction, NJ (US)

(72) Inventor: Niral Ramesh, Princeton Junction, NJ (US)

(21) Appl. No.: 13/951,290

(22) Filed: Jul. 25, 2013

(51) Int. Cl. A61K 9/00; A23L 1/30

(52) U.S. Cl. CPC A61K 9/0056; A61K 9/0095; A23L 1/3008
USPC 514/560

(57) ABSTRACT

A grain free solid-in-oil dispersion comprising a medium chain fatty acid (MCFA), at least one polyunsaturated fatty acid (PUFA), and at least one solid, is provided. The dispersion compositions are useful for preparing dosage forms, dietary supplements and foods that provide health benefits.

### FIELD OF THE INVENTION

The present invention relates to a grain free solid-in-oil dispersion having a medium chain fatty acid (MCFA), at least one polyunsaturated fatty acid (PUFA), and at least one solid, wherein the dispersion is useful a grain free food product that provides health benefits.

### BACKGROUND OF THE INVENTION

It has been proposed that the western diet is one of the primary drivers of increasing chronic diseases such as fatty liver diseases, obesity, diabetes, cancer, epilepsy, multiple sclerosis, gastroesophageal reflux disease and inflammatory diseases. The pathophysiology and genetics of these complex disorders are slowly being unraveled. Currently no effective remedies are available for treating most of these disorders. One strategy currently used in clinical medicine is metabolic manipulation with health food products to reduce and or prevent the chronic diseases outlined above. Developing novel food products requires redesigning the extent and type of calories, extent and type dietary fuels, nutrients, and consumption timings of food products to achieve the desired effects.

The biochemical pathways for synthesis and breakdown of the three major metabolic fuels, carbohydrates, fats, and protein, converge in the liver. In mammals, the liver is one of the few organs that can carry out almost all of the various pathways associated with these three dietary agents.

Carbohydrate, beyond its role as a source of energy, has an important regulatory function. Dietary carbohydrate stimulates insulin secretion, and/or affects the availability of energy substrates such as free fatty acids, ketone bodies and glycogen. Carbohydrates also provide a direct source of glucose or fructose, both of which can serve as stimuli and regulators. It has been reported that glucose-affects genes that include the primary lipogenic enzymes acetyl-CoA carboxylase (ACC) and fatty acid synthase (FAS). Carbohydrate response element binding protein (ChREBP) has provided evidence for carbohydrate is responsible for activation of lipogenic genes. High glucose levels are reported to activate ChREBP modulating liver pyruvate kinase, ACC, and stimulation of FAS transcription without any evidence that glucose directly regulates nutrient partitioning.

It has also been reported that Insulin affects expression of more than 150 genes and that amplification of insulin signaling has diverse effects, such as, Glycogenolysis (Glycogen breakdown), Gluconeogenesis (Glucose synthesis), Lipolysis, (Lipid breakdown), glycogen storage and protein synthesis. At the cellular level, it has been reported that the carbohydrate-insulin axis is coordinated through two interacting pathways involving the ChREBP and a sterol response element binding protein 1c (SREBP-1c). Insulin has also been reported to modulate a key regulator of lipid metabolism, liver X receptor (LXR), a nuclear hormone receptor. Oral administration of LXR agonists reportedly can lead to increased hepatic de novo lipogenesis (DNL), steatosis, VLDL and hypertriglyceridemia.

Fructose, a monosaccharide, is another regulator that has been reported to be directly modulated by dietary carbohydrate. Increasing fructose intake, which has been estimated at about 85-100 grams/day, can exposes the liver to high levels of this monosaccharide, which can by-pass the regulatory control step at phosphofructose kinase-1, increasing dihydroxyacetone phosphate, and can lead to increasing glycerol-3-phosphate for TG synthesis. Thus, fructose ingestion has been associated with a general disruption in fuel metabolism and can acutely enhance postprandial lipemia and metabolic syndrome.

Ketone bodies, produced in hepatocytes, are also very sensitive to carbohydrate levels. Lipid metabolism, via mitochondrial β-oxidation, e.g., during fasting, has been reported to be due to the specific removal of carbohydrates as opposed to a general elimination of calories. Ketogenic (high fat) diets have long been known to increase activation of genes in lipid oxidation and decreased expression of genes in lipogenes. Ketogenic diets have been shown to induce hepatic expression and increase circulating levels of fibroblast growth factor 21 (FGF21). FGF21 has been found to be a critical modulator in liver, coordinating lipid homeostasis, which is believed to occur by inducing hepatic lipid oxidation, ketogenesis, white adipose tissue lipolysis, and TG clearance.

Carbohydrate consumption influences glycogen, which has historically been viewed in the narrow context as a substrate, especially during skeletal muscle contraction. Recently, reports have linked glycogen metabolism with a range of metabolic processes, including glucose transport into cells. Several experimental models have shown that muscle glycogen levels exert an important influence on insulin-mediated and contraction-mediated glucose uptake, as well as basal, un-stimulated, glucose entry into cells. Glucose uptake is higher in glycogen-depleted muscle and there appears to be an inverse relationship between glycogen and glucose uptake across a broad range of glycogen levels. Further, support for the proposal that glycogen acts as an energy regulator comes from the fact that exercise is believed to induce an increase in skeletal muscle GLUT4, an insulin-dependent glucose transporter, and a proportional increase in glucose transport capacity. Carbohydrates fed after exercise are believed to increase glucose entry into cells and promote glycogen formation. As glucose enters into the cells, and glycogen levels increase, glucose transport is inhibited and there is a return of GLUT4 to pre-exercise levels. Prevention of glycogen synthesis, by fasting or feeding a low-carbohydrate/high-fat diet, results in a persistence of contraction and insulin-mediated glucose transport that lasts as long as carbohydrates are not consumed. This is believed to mean that low glycogen promotes increased insulin action, and high glycogen promotes insulin resistance.

Additional support for the proposal that carbohydrates can acts as energy regulators as well as an energy source is found in studies reporting that even a short-term reduction in carbohydrate levels can cause effects on transcriptional control of many genes. Reducing a subject's carbohydrate intake from 49 to 34% of energy for 3 days has been reported to differentially regulate over 300 genes in skeletal muscles in a manner consistent with a shift from glucose utilization to fatty acid utilization, via oxidation. In 15 studies, experiments comparing low-fat and low-carbohydrate diets show greater increases in HDL-C on carbohydrate-restricted diets compared to low-fat diets with an average absolute difference of 11%.

Carbohydrate restriction, even in the presence of high concentration of saturated fatty acids, appears to decrease the availability of ligands (such as glucose, fructose, and insulin) that can activate lipogenic gene expression and inhibit fatty acid oxidative pathways. Although the specific carbohydrate affected transcriptional pathway is unclear, the result appears clear, an increase in fat oxidation, a decrease in lipogenesis and a decrease in secretion of very low-density lipoprotein. Dietary carbohydrate perturbs upstream response elements that result in dysfunctional metabolic state orchestrated through several key transcription factors. the proposal that a high carbohydrate diet can drive dysfunction is grounded in basic metabolic and evolutionary principles. A carbohydrate restricted intake is an evolutionarily preferred dietary strategy for managing modern diseases, rather than fat restrictions. Thus, the risks of at least 35 chronic health conditions have increased.

It has been hypothesized that excess carbohydrate overloading can leads to energy dysfunction and/or oxidative stress because of the disturbance to major metabolic processes like oxidative phosphorylation, gluconeogenesis, glycolysis, glycogenesis, glycogenolysis, fatty acid biosynthesis, fatty acid oxidation, ketogenesis, protein synthesis, amino acid cycles, PPP, TCA cycle, urea cycle, BAA cycle, etc. are all self-regulatory and in equilibrium. Metabolomic studies implicate dysfunctional energy metabolism in multiple diseases, such as, retinopathy, schizophrenia, MDD, Bipolar disorder, IBD and cancer. Thus, the research data indicate dietary carbohydrate in western diet is a key cause of metabolic disorders. Manipulation of the extent and type of dietary carbohydrates contained in food and health products is a crucial step in developing novel these products to assist in maintaining the health of the population. Thus, redesigned health foods can help persons affected with modern diseases.

Fatty acids (FAs) constitute an important source of energy in humans not only during fasting but also under well-fed conditions, since some organs, including the heart, show a marked preference for FAs at all times. Fatty acids are classified as short-chain (<C₇) fatty acids (SCFAs), medium-chain (C₈-C₁₂) fatty acids (MCFAs), long-chain (C₁₃-C₂₀) fatty acids (LCFAs) and very long chain fatty acids (VLCFAs).

Dysfunctional fatty acid metabolism is reported to be central in many chronic diseases such as cardiovascular disease, diabetes, cancer, degenerative eye and brain diseases, psychiatric, fatty liver diseases and inflammatory diseases. Mitochondrial fatty acid oxidation (FA-O) is the principal pathway for oxidation of FAs, although FAs can also undergo alpha- and omega-oxidation. The alpha- and omega-oxidation pathways do not contribute much to the oxidation of FAs in terms of energy production in human beings and depend on beta-oxidation for further degradation of the FAs. Importantly, in higher eukaryotes including humans, beta-oxidation not only occurs in mitochondria but also occurs in peroxisomes. Both mitochondrial and peroisomal oxidation proceed via a similar mechanism involving four enzymatic steps in which an acyl-coenzyme A ester (acyl-CoA) undergoes subsequent steps of dehydrogenation, hydratation, a second dehydrogenation, and finally termination with thiolytic cleavage. Despite these similarities, there are major differences between the two systems in terms of the enzymes involved, their regulation, and the substrates handled by the two oxidation systems. There are reports that the bulk of the dietary FAs including palmitic acid, oleic acid, and linoleic acid are beta-oxidized in mitochondria. Peroxisomes, however, play an equally indispensable role in whole cell fatty acid oxidation, by catalyzing the beta-oxidation of a range of FAs and fatty acid derivatives that are not handled by mitochondria, which include very-long-chain FAs, pristanic acid, and the bile acid intermediates di- and tri-hydroxycholestanoic acid. The distinct physiological roles of the two beta-oxidation systems is exemplified by the differences in clinical signs and symptoms of patients affected by either a mitochondrial beta-oxidation defect or a peroxisomal beta-oxidation defect.

The dominant mitochondrial β-oxidation pathway, for the disposal of fatty acids under normal physiologic conditions, primarily involves the oxidation of short-chain (<C₇) fatty acids (SCFAs), medium-chain (C₈-C₁₂) fatty acids (MCFAs) and long-chain (C₁₃-C₂₀) fatty acids (LCFAs). Very long chain fatty acids (VLCFAs) cannot undergo mitochondrial β-oxidation. Short-chain and medium-chain FFAs can freely enter the mitochondria, while long-chain FFAs entry into the mitochondria is regulated by the activity of the enzyme carnitine palmitoyl transferas (CPT-I).

Triacylglycerols containing fatty acids with more than 12 carbon atoms must be hydrolyzed in the intestinal lumen before absorption. In the enterocytes they will be re-synthesized to triacylglycerols and preferentially used for the formation of chylomicrons. LCFA are released from the chylomicrons by serum lipases and can be metabolized by tissue or stored in the adipocytes. Triacylglycerols containing C₈-C₁₂ carbon chains can directly be absorbed without hydrolysis and preferentially transported through the portal venous system to the gastrointestinal system. On the hepatocellular level, the carnitine acyltransferase, the enzyme system necessary for C₁₃₋₂₀ carbon chains transport across the inner mitochondrial membrane, is not required for MCFA. Thus, C₈-C₁₂ fatty acids are more available for the mitochondrial β-oxidation whereas most C₁₃₋₂₀ fatty acids are incorporated into triacylglycerols in the hepatocyte. As C₈₋₁₂ fatty acids bypass the adipose tissue lipoprotein lipase-regulated metabolic pathway of long chain fatty acids, the lipid deposition into adipocytes may be limited by dietary medium chain fatty acid intervention. Additionally, it has been reported that dietary intervention with C₈₋₁₂ carbon chain fatty acids increased thermogenesis and endogenous oxidation of C₁₃₋₂₀ fatty acids.

Omega-3-polyunsaturated fatty acids (PUFAs) are fatty acids with hydrocarbon chains with two or more double bonds situated along the length of the carbon chain. Depending on the location of the first double bond relative to the methyl terminus, they can be classified as either n-6 or n-3. Polyunsaturated fatty acids represent the fundamental components of phospholipids in cellular membranes. PUFAs are usually located in the sn-2 position, whereas saturated or monounsaturated fatty acids are usually bound in the sn-1 position of the phospholipid molecules. Fatty acids integrated in these positions are the healthy food of dietary fat. In recent years, there has been increased focus on the role of specific dietary fatty acids, particularly polyunsaturated fatty acids, and their effect on health and disease. Linoleic acid (LA; 18:2n-6), the parent fatty acid of the n-6 PUFA family is an essential fatty acid and cannot be endogenously synthesized by mammals. LA is found in vegetable oils, seeds and nuts. Alpha-linolenic acid (ALA; 18:3n-3), the parent fatty acid of the n-3 PUFA family, must be consumed through the diet. ALA can be found in leafy vegetables, walnuts, soybeans, flaxseed, and seed and vegetable oils. Both LA and ALA can be further metabolized to long chain PUFA through a series of desaturation and elongation steps. LA is metabolized to arachidonic acid (AA, 20:4n-6), while ALA can be metabolized to eicosapentaenoic acid (EPA; 20:5n-3) and ultimately docosahexaenoic acid (DHA; 22:6n-3). Alternatively, AA can be obtained from animal fat sources and EPA and DHA can be consumed directly from marine sources. Both ALA and LA are converted to their respective long chain metabolites by the same set of enzymes. However, the metabolic products of each pathway are structurally and functionally distinct. The metabolites of n-6-long chain PUFAs are pro-inflammatory while those of n-3 long chain PUFAs are anti-inflammatory. Thus the high potency of PUFAs, particularly n-3 PUFA, to regulate metabolism may reflect the formation of their metabolites acting as signaling molecules.

Further, reports indicate that dysfunctional fatty acid metabolism is a critical converging factor in all these diseases. One approach to modulate the energy homeostasis is modulation of fatty acid metabolism by redesigning foods that have specific fatty acids that provide a specific level of calories.

It has been reported that inflammation is often chronically present in many of these disorders characterized by elevated levels of cytokines, particularly Interleukin-6 and Tumor Necrosis Factor.

Interleukin-6, an inflammatory cytokine, is characterized by pleiotropy and redundancy of action. IL-6 is a 26-kDa glycopeptide, whose gene is found on chromosome 7. It has previously been known as hepatocyte-stimulating factor, cytotoxic T-cell differentiation factor, B-cell differentiation factor, B-cell stimulatory factor 2, hybridoma/plasmacytoma growth factor, monocyte granulocyte inducer type 2 and thrombopoietin.

IL-6 has many endocrine and metabolic actions in addition to its hematologic, immune, and hepatic effects. Specifically, it is a potent stimulator of the hypothalamic-pituitary-adrenal axis and is under the tonic negative control of glucocorticoids. It acutely stimulates the secretion of growth hormone, inhibits thyroid-stimulating hormone secretion, and decreases serum lipid concentrations. Furthermore, it is secreted during stress and is positively controlled by catecholamines. Administration of interleukin-6 results in fever, anorexia, and fatigue. Elevated levels of circulating interleukin-6 have been seen in the steroid withdrawal syndrome and in the severe inflammatory, infectious, and traumatic states potentially associated with the inappropriate secretion of vasopressin. Interleukin-6 is negatively controlled by estrogens and androgens, and it plays a central role in the pathogenesis of the osteoporosis, seen in conditions characterized by increased bone resorption, such as, sex-steroid deficiency and hyperparathyroidism. Overproduction of interleukin-6 may contribute to illness during aging and chronic stress. Levels of circulating interleukin-6 are reported to be elevated in several liver diseases. Serum IL-6 levels are elevated in animal models and in patients having non-alcoholic fatty liver disease (NAFLD), Alcoholic fatty liver disease and primary biliary cirrhosis. It has recently been reported that IL-6 is up-regulated in the liver of two well-established murine models of NAFLD. In human liver diseases, IL-6 expression is increased in both hepatocytes and Kupffer cells and the levels positively correlate with both the inflammatory activity and the stage of fibrosis.

Similarly, extensive evidence supports a central role of another pro-inflammatory cytokine, tumor necrosis factor-alpha (TNF-α). This pro-inflammatory cytokine mediates the development of insulin resistance, fatty liver diseases, psychiatric, neurodegenerative and inflammatory diseases. Circulating, as well as liver and adipose tissue, levels of TNF-α are increased in obesity and fatty liver disease animal models. Administration of TNF-α to culture cells in vivo or to animals is believed to impair insulin action. Animals lacking TNF-α or TNF-α receptors, have improved insulin sensitivity in both dietary and genetic obesity models. This is also reported in humans, where TNF-α levels correlate with the degree of insulin resistance. Furthermore, in humans, acute infusion of TNF-α inhibits insulin-stimulated glucose disposal, and certain TNF-α polymorphisms are associated with susceptibility to insulin resistance and NAFLD, supporting the importance of this cytokine in the interaction among inflammation, insulin signaling, and fat accumulation.

The prevention and/or reduction of inflammation by using dietary ingredients that lower IL-6 and TNF is a useful approach to slow the progression of these chronic diseases. Thus, global synergistic modulation of fatty acid oxidation, carbohydrate and protein intake, homeostasis and inflammation with one health food product platform provides an approach to develop very beneficial supplements and food that enhance health benefits. It is especially useful if a health food product is optimally designed to manage the fatty acid metabolism, energy imbalance and inflammation.

Omega-3-fatty acids are known to provide several benefits. The effect of n-3 long chain PUFAs depend on the ratio of omega n-6 to omega n-3 PUFAs. The scientific literature does not disclose a unique ratio of omega n-6 to omega-3 PUFAs that can provide synergistic action. Further, the literature does not disclose the ability of omega-3 fatty acid preparations to provide synergistic action that depends on their relative content of EPA and DHA, as well as the purity of the overall formulation and also on the presence and amount of a medium chain fatty acid (MCFA) and a protein.

The use of a mixture of docosahexanoic acid, eicosapentaenoic acid and gamma-linolenic acid (an ω-6 fatty acid) for the treatment of cancer is disclosed in EP 0 175 468. U.S. patent application 2010/0323982 discloses a solid-in-oil dispersion comprising (a) one or more ω-3 fatty acids selected from DHA, DPA and EPA, (b) uridine or its equivalent, and (c) a methyl donor, useful in the treatment of a person having characteristics of a prodromal dementia patient. EP 1 216 041 discloses the use of a high amount (35-70 en %) of a lipid blend with 25-70 wt % MCT and ω-6/ω-3=2-7:1 in the manufacture of a food product for the treatment of sepsis or inflammatory shock. WO 2003/013276 discloses the use of 30-70% of a lipid blend, which is rich in oleic acid (50-70 wt %) and specific amounts of ω-6 fatty acids and 1-10 wt % ω-3 fatty acids for increasing intramyocellular lipid levels in muscle cells. EP 0 175 468 A2 provides a method of normalizing cellular eicosanoid balance by administering to a warm blooded animal an effective amount of a solid-in-oil dispersion chosen from the group comprising eicosapentanoic acid (EPA), docosahexanoic acid (DHA) a mixture of EPA and DHA and a mixture of EPA, DHA and GLA.

U.S. Pat. No. 5,886,037 discloses a composition suitable for the treatment of increased plasma lipid levels in hypertriglyceridaemia or hyperchylomicronaemia, comprising fats, the fatty acids of said fats comprising: 55-95 wt. % of medium chain fatty acids (MCFA's); 5-25 wt. % of n-3 polyunsaturated fatty acids (n-3 PUFA's); and 0-30 wt. % of other fatty acids. U.S. Pat. No. 8,283,335 discloses a lipid fraction that comprises the medium-chain fatty acids at least 4 g hexanoic acid and/or at least 5 g octanoic acid, at least 1 g eicosapentaenoic acid, and in addition more than 0.4 g of alpha-linolenic acid per 100 g fatty acids of the lipid fraction. U.S. Pat. No. 7,560,486 discloses an isotonic lipid-in-water emulsion free of long-chain vegetable oils, said emulsion comprising (i) 60 to 95% by weight of medium chain triglycerides (MCT), and (ii) 5 to 40% by weight of fish oil, based on the total amount by weight of MCT and fish oil lipids in the emulsion. U.S. Pat. No. 8,241,672 discloses an oil-in-water emulsion of enriched fish oil and a medium chain fatty acid (MCFA) (MCT) oil, wherein the enriched fish oil comprises at least 45% by weight of EPA and DHA, and at least 60% by weight of n3-FA, based on the total weight of the enriched fish oil.

While not wishing to be bound by theory, it is believed that the omega-3 polyunsaturated fatty acids work by acting at different sites and aspects by modulating energy homeostasis, fatty acid oxidation and inflammation reduction to provide health benefits. Similarly, individual medium chain fatty acids (MCFAs) have activities that depend on the type of specific MCT and extent of its purity. These complex sets of modulations are likely influenced not only by the relative ratios of polyunsaturated fatty acids, but also by the ratios of medium chain fatty acid (MCFAs) between themselves and with polyunsaturated fatty acids. The literature does not disclose a grain free solid-in-oil dispersion comprising a medium chain fatty acid (MCFA) and at least one polyunsaturated fatty acid (PUFA), at least one solid which is at least 50% by weight of total solid-in-oil dispersion, wherein the a medium chain fatty acid (MCFA) and PUFA provide synergistic action. It would extremely valuable if it is possible to develop a common dietary solid-in-oil platform technology that is useful for preparing grain free supplements and food products that reduce or prevent chronic and age related diseases with minor modifications.

It has been discovered that a grain free solid-in-oil dispersion comprising a medium chain fatty acid (MCFA), at least one polyunsaturated fatty acid (PUFA), at least one solid which is at least 50% by weight of total solid-in-oil dispersion, can be used as a platform formulation to prepare supplements and foods that provides surprisingly useful dietary and health benefits, both alone and in combination with other agents, in reducing or preventing several chronic disorders is disclosed herein. The disclosed composition provides a fortified solid-in-oil dispersion having one or more of the disclosed solid-in-oil dispersed composition that enable preparation of dosage forms, supplements, and foods that provide health benefits.

There is an unmet need for more efficient solid-in-oil dispersions to prepare a dosage form, a dietary supplement or a food product that reduces or prevents chronic and age related diseases. It will be especially helpful to find a common platform formulation useful as dosage forms, supplements and food products to reduce or prevent these diseases, either alone or in combination with another dietary agent. In addition, there is a need for high efficacy nutritional solid-in-oil dispersions that help prepare dietary supplements and food products that provide benefits for patients with cardiovascular, diabetic, neurodegenerative, eye, psychiatric, liver and inflammatory disorders, without undesirable side effects. Further, there is an unmet need for nutritional solid-in-oil dispersions and/or forms for preparing supplements and food products to reduce or prevent diabetic, psychiatric, neurodegenerative, eye, and inflammatory disorders that are free from or have limited side effects associated with physician prescribed medications. Thus, global synergistic modulation of fatty acid oxidation, homeostasis and inflammation with one solid-in-oil dispersion platform can provide a novel approach to develop novel supplements and foods that enhance health benefits.

### SUMMARY OF THE INVENTION

In one aspect, the present invention provides a grain free solid-in-oil dispersion comprising at least one medium chain fatty acid (MCFA), at least one polyunsaturated fatty acid (PUFA), and at least one solid, wherein the solid is at least about 50% by weight of the dispersion, and optionally a dietary carrier, or a preservative agent. The dispersion is useful to prepare dosage forms, dietary supplements and food products.

In another aspect, the present invention provides a grain free solid-in-oil dispersion comprising from about 35% to about 90% a medium chain fatty acid by weight of total fat in the dispersion, from about 10% to about 65% of at least one polyunsaturated fatty acid (PUFA) by weight of total fat in the dispersion and at least one solid, or acceptable salts thereof, and optionally a dietary carrier or a preservative agent. The solid can be a powder, such as an isolated protein, a flour, or acceptable salts thereof, and is at least about 50% by weight of the dispersion composition.

The solid-in-oil dispersion derives part or all of its medium chain fatty acid from a whole food such as coconut flour, palm drupe flour, camphor drupe flour, and the like.

In another aspect, the present invention provides a grain free solid-in-oil dispersion wherein the polyunsaturated fatty acid (PUFA) is eicosapentaenoic acid (EPA). Preferably, the solid-in-oil dispersed composition contains not more than about 10%, by weight, of docosahexaenoic acid.

In another aspect, the present invention provides a grain free solid-in-oil dispersion wherein the polyunsaturated fatty acid (PUFA) is docosahexaenoic acid. Preferably, the solid-in-oil dispersed composition contains not more than about 10%, by weight, of eicosapentaenoic acid (EPA).

In another aspect, the present invention provides a grain free solid-in-oil dispersion wherein the medium chain fatty acid (MCFA), is caprylic acid. Preferably, the solid-in-oil dispersed composition contains not more than about 10%, by weight, of other medium chain fatty acid (MCFAs).

In another aspect, the present invention provides a nutraceutical composition comprising a grain free solid-in-oil dispersion having from about 35% to about 90% a medium chain fatty acid by weight of total fat in the dispersion, from about 10% to about 65% of at least one polyunsaturated fatty acid (PUFA) by weight of total fat in the dispersion and at least one solid, or acceptable salts thereof, and optionally a dietary carrier or a preservative agent. The solid is at least about 50% by weight of the dispersion composition. The solid-in-oil dispersion derives part or all of its medium chain fatty acid from a whole food such as coconut flour, palm drupe flour, camphor drupe flour, and the like.

In another aspect, the present invention the nutraceutical composition has from about 35% to about 90% by weight of capric acid. The solid-in-oil dispersed composition contains not more than about 10%, by weight, other medium chain fatty acid (MCFAs).

In another aspect, the present invention provides a grain free solid-in-oil dispersion, wherein the dispersion is suitable for admixing with a food product such as a breakfast cereal snacks, drink, an ice cream, a meal, or a dessert.

In another aspect, non-limiting examples of the polyunsaturated fatty acid include but are not limited to ethyl eicosapentaenoic acid (Ethyl EPA), linolenic acid (LA), arachidonic acid (AA), docosahexaenoic acid (DHA), alpha-linolenic acid (ALA), conjugated linoleic acid, stearadonic acid (STA), eicosatrienoic acid (ETA), docosapentaenoic acid (DPA), or nutraceutically acceptable salts or derivatives thereof.

In another aspect, solid-in-oil dispersions disclosed, an optional additional dietary agent such as, for example, a vitamin, an amino acid, a hormone, an element, a nutrient, and the like.

The details of one or more embodiments of the invention are set forth in the accompanying description below. Other features, objects, and advantages of the invention will be apparent from the description and drawings, and from the claims.

### DESCRIPTION OF THE FIGURES

FIG. 1 illustrates the change in serum triglyceride concentration from baseline to end of study.

FIG. 2 illustrates the change in serum LDL concentration from baseline to end of study.

FIG. 3 illustrates the change in serum HDL concentration from baseline to end of study.

FIG. 4 illustrates the percent change in the mean serum concentrations of β-hydroxy butyrate.

FIG. 5 illustrates the change in the mean serum concentrations of IL-6.

FIG. 6 illustrates the change in the mean serum concentrations of TNF.

FIG. 7 illustrates the change in the mean serum concentrations of 25-hydroxy vitamin D.

### DETAILED DESCRIPTION

### Definitions

The following terms have the meaning as defined in this section. Although any materials and methods similar or equivalent to those described herein can be used in the practice or testing of the present invention, the preferred materials and methods are described herein. In describing and claiming the invention, unless otherwise defined, all technical and scientific terms used herein have the same meaning as commonly understood by one of ordinary skill in the art to which this invention belongs. Exemplary and preferred values listed below for radicals, substituents, and ranges are for illustrations only; they do not exclude other defined values or other values within defined ranges for the radicals and substituents.

The terms “a,” “an,” “the,” “at least one,” and “one or more” are used interchangeably. Thus, for example, a grain free solid-in-oil dispersion that comprises “an” element means one element or more than one element.

The term “additive effect” as used herein means the effect resulting from the sum of the effects obtained from the individual agents.

The term “capric acid” or “capric triglyceride” used herein means the capric acid and its salts and esters of the saturated fatty acid with CH₃(CH₂)₈COOH including capric triglyceride.

The term “caprylic acid” or “caprylic triglyceride” used herein means the salts and esters of the saturated fatty acid with CH₃(CH₂)₆COOH including caprylic triglyceride.

The term “clinical benefits” or “clinical benefit” as used herein means improvement in the clinical symptoms associated with a disease in at least 30% of the patients upon administration of medication as determined by clinician using a clinically accepted measure.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “effective amount” as used herein, means an amount sufficient to produce a selected effect using an amount that is sufficient to prepare a dietary supplement, a food, or drug to provide an effective food, supplement, or drug. For example, for the polyunsaturated fatty acid present in the disclosed grain free solid-in-oil dispersion an effective amount depends on the total fat, type of fat, type of powder present in solid-in-oil dispersed composition and the food, supplement and drug used to prepare them. Similarly, the effective amount of a medium chain fatty acid (MCFA) depends on the total fat, type of fat, type of powder present in solid-in-oil dispersed composition and the food, supplement and drug used to prepare them.

The term “grain” or “grains” as used herein means food grains from Monocotyledons (also known as monocots) family of flowering plants. Monocot seedlings typically have one cotyledon (seed-leaf), in contrast to the two cotyledons typical of dicotyledons or dicots. They include maize, rice, wheat, barley, millet, oats, rye, ragi, jawar, and the like. Thus, a grain free solid-in-oil dispersion will be free from products derived from these monocotyledon plants.

The term “health benefits” or “health benefit” as used herein means reducing the risk of occurring of a disorder or reduction in the symptoms of disorder present in a mammal.

The term “IL-6” or “Interleukin-6” as used herein means the IL-6 cytokine T cells and macrophages secret this cytokine to stimulate an immune response to trauma, particularly burns or other tissue damage leading to inflammation.

The term “ketone body” as used herein means one of the three endogenous ketone bodies, acetone, acetoacetic acid, and beta-hydroxybutyric acid. A ketone body is released when the fatty acids are metabolized in the mitochondria.

The term “medium chain fatty acid (MCFA)” or “medium chain triglyceride” or “medium chain saturated fatty acid” means linear or branched saturated carboxylic acids having four, five, six, seven, eight, nine, ten, eleven, or twelve carbon atoms either in free acid form or in their respective sales, esters including as a triglyceride.

The term “medicament,” “drug,” or “active agent” as used herein, means a polyunsaturated fatty acid (PUFA) or a medium chain fatty acid (MCFA) or third active agent used in the solid-in-oil dispersed composition or an acceptable form, e.g., their respective optically active enantiomers, racemic mixtures thereof, pro-agents (pro-drugs), active metabolites, and/or acceptable salts thereof, such as, for example, acid addition or base addition salts of an active agent.

The term “monoagent use” as used herein, means the use of only one active ingredient for the preparation of the food composition.

The term “nutraceutically acceptable derivative” or “acceptable derivative” as used herein, means various equivalent isomers, enantiomers, complexes, salts, hydrates, polymorphs, esters, and the like, e.g., of an active agent—a polyunsaturated fatty acid (PUFA) a medium chain fatty acid (MCFA).

The term “nutraceutically acceptable salt” or “acceptable salt” as used herein, refers to salts that retain the biological effectiveness and properties of polyunsaturated fatty acid (PUFA), and or a medium chain fatty acid, which are not biologically or otherwise undesirable.

The term “Polyunsaturated Fatty Acid” or “PUFA” includes but is not limited to omega-3 unsaturated fatty acids such as α-linolenic acid, stearidonic acid, eicosapentaenoic acid and docosahexaenoic acid, omega-6 unsaturated fatty acids such as conjugated linoleic acid, linoleic acid, γ-linolenic acid, dihomo-γ-linolenic acid, arachidonic acid, and adrenic acid, omega-7 unsaturated fatty acids such as palmitoleic acid, vaccenic acid, and paullinic acid and omega-3 unsaturated fatty acids such as oleic acid, elaidic acid, gondoic acid, erucic acid, nervonic acid and mead acid or nutraceutically acceptable salts thereof.

The term “prevention of a disease” or “prevention of a disorder” as used herein, is defined as the management and care of an individual at risk of developing the disease prior to the clinical onset of the disease. The purpose of prevention is to combat the development of the disease, condition or disorder, and includes the administration of the active agents to prevent or delay the onset of the symptoms or complications and to prevent or delay the development of related diseases, conditions or disorders.

The term “saturated fatty acid” as used herein means saturated fatty acids esters of glycerol having a carbon chain with from 2 to about 36 carbon atoms, preferably having a carbon chain with from 2 to about 22 carbon atoms. Non-limiting examples of the saturated fatty acids include propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid.

The term “solid” or “powder” as used herein includes insoluble drugs, isolated proteins, or flours. Non-limiting examples of protein sources include milk, soy, whey, wheat, tofu, collagen, albumin, gelatin, caseinates, peas, hemp and rice protein. Non-limiting examples of insoluble drugs include itraconazole, etravirine, tacrolimus, rosuvastatin, griseofulvin, nabilone and the like. Non-limiting examples of flours include almond flour, coconut flour, alfalfa flour, and the like but exclude grain flours.

The term “synergistic effect” as used herein means an effect from two agents (active agents), which is greater than the additive effect that results from the sum of the effects of the two individual agents.

The term “TNF” or “TNF-alpha (TNF-α)” or “Tumor Necrosis Factor-α” or “Cachectin” or “cachexin” as used herein means the macrophage-secreted cytokine.

The term “reducing or preventing a disease” as used herein, means the preventive management and care of a mammal against a disease, condition or disorder.

The amount of the disclosed solid-in-oil dispersed composition to be administered to the patient can vary. Suitable amounts are well known to those skilled in the art. Factors such as, for example, the weight of the patient, the route of administration, or the severity of the illness can affect the exact amount administered.

### Solid-in-Oil Dispersions

The present invention provides grain free solid-in-oil dispersions comprising a medium chain fatty acid (MCFA), at least one polyunsaturated fatty acid (PUFA), or a nutraceutically equivalent salt thereof and at least one solid wherein the dispersion provides health benefits.

In one embodiment, the present invention provides solid-in-oil dispersions comprising a medium chain fatty acid (MCFA), at least one polyunsaturated fatty acid (PUFA), or a nutraceutically equivalent salt thereof and at least one solid. The disclosed solid-in-oil dispersions can be used in single serving or multiple servings to a subject in need thereof. The preferred polyunsaturated fatty acid is eicosapentaenoic acid and the solid-in-oil dispersed composition contains not more than about 10%, by weight, of docosahexaenoic acid.

In another embodiment, the present invention provides solid-in-oil dispersions comprising a medium chain fatty acid (MCFA), at least one polyunsaturated fatty acid (PUFA), or a nutraceutically equivalent salt thereof and at least one solid. The disclosed solid-in-oil dispersions can be used in single dose/servings or multiple doses/servings to a subject in need thereof. The preferred polyunsaturated fatty acid is docosahexaenoic acid and the solid-in-oil dispersed composition contains not more than 10%, by weight, of eicosapentaenoic acid.

In another embodiment, the present invention provides the use of at least one omega-3 polyunsaturated fatty acids (PUFA). Preferably, the solid-in-oil dispersed composition comprises at least one PUFA selected from docosahexaenoic acid (22:6 ω-3; DHA), docosa-pentaenoic acid (22:5 ω-3; DPA) and eicosapentaenoic acid (20:5 ω-3; EPA). More preferably, the present solid-in-oil dispersed composition contains at least DHA, preferably DHA and EPA. Even more preferably, the solid-in-oil dispersed composition contains DHA and at least one precursor of DHA selected from EPA and DPA. Still more preferably, the present solid-in-oil dispersed composition comprises DHA, DPA and EPA.

A further aspect the present solid-in-oil dispersed composition contains a substantial amount of EPA. EPA is converted to DPA (ω-3), increasing subsequent conversion of DPA (ω-3) to DHA in the brain. Hence, the present solid-in-oil dispersed composition preferably also contains a significant amount of EPA, to stimulate in-vivo production of DHA.

In another embodiment, the solid-in-oil dispersions comprises PUFA that is preferably free fatty acids or their salts or esters, phospholipids, lysophospholipids, ethers, glycolipids, lipoproteins, ceramides triglycerides, diglycerides, monoglycerides, or combinations thereof.

In another embodiment, the present solid-in-oil dispersed composition provides from about 400 to about 10,000 mg (of PUFA, DHA+EPA) per day, preferably an amount of from about 500 to about 4000 mg per day, and more preferably from about 500 to about 3000 mg per day. The disclosed amounts take into account and optimize several factors such as taste, and balance between DHA and precursors thereof, thus ensuring optimal effectiveness and maximum dosage in the product formulations, such as, liquid form, bar or capsule.

In another embodiment, a grain free solid-in-oil dispersion comprising eicosapentaenoic acid is provided containing less than about 10%, preferably less than about 7%, even more preferably less than about 4%, still even more preferably less than about 1%, and most preferably less than about 0.35%, by weight of the total weight of the fatty acid content, of any fatty acid other than EPA. Non-limiting examples of a “fatty acid other than EPA” include linolenic acid (LA), arachidonic acid (AA), docosahexaenoic acid (DHA), alpha-linolenic acid (ALA), stearadonic acid (STA), eicosatrienoic acid (ETA) and/or docosapentaenoic acid (DPA).

In another embodiment, a grain free solid-in-oil dispersion comprising docosahexaenoic acid (DHA) provided containing less than about 10%, preferably less than about 7%, even more preferably less than about 4%, still even more preferably less than about 1%, and most preferably less than about 0.35%, by weight of the total weight of the fatty acid content, of any fatty acid other than DHA. Non-limiting examples of a “fatty acid other than DHA” include linolenic acid (LA), arachidonic acid (AA), eicosapentaenoic acid (EPA), alpha-linolenic acid (ALA), stearadonic acid (STA), eicosatrienoic acid (ETA) and/or docosapentaenoic acid (DPA).

In another embodiment, the disclosed grain free solid-in-oil dispersion has one or more of the following features: (a) the amount of eicosapentaenoic acid ethyl ester in the composition represents at least about 97% by weight, preferably at least about 96%, or more preferably at least about 90%, by weight, of the total of all fatty acids present in the solid-in-oil dispersed composition; (b) the composition contains not more than about 10%, preferably not more than about 4%, or most preferably not more than about 3%, by weight, of total fatty acids other than eicosapentaenoic acid ethyl ester; (c) the composition contains not more than about 0.6% by weight, preferably not more than about 0.5%, or more preferably not more than about 0.10% of any individual fatty acid other than eicosapentaenoic acid ethyl ester; (d) the composition has a refractive index (20° C.) of from about 1 to about 2, preferably from about 1.2 to about 1.8 or more preferably from about 1.4 to about 1.5; (e) the composition has a specific gravity (20° C.) of from about 0.8 to about 1.0, preferably from about 0.85 to about 0.95 or more preferably from about 0.9 to about 0.92; (f) the composition contains not more than about 20 ppm of heavy metals, preferably more than about 15 ppm of heavy metals or more preferably more than about 10 ppm of heavy metals; (g) the composition contains not more than about 5 ppm of arsenic, preferably not more than about 4 ppm of arsenic, more preferably not more than about 3 ppm of arsenic, or most preferably not more than about 2 ppm of arsenic; and (h) the composition has a peroxide value not more than about 5, preferably not more than about 4, more preferably not more than about 3, or most preferably not more than about 2 meq/kg.

In another embodiment, the present solid-in-oil dispersed composition preferably contains a low amount of arachidonic acid (AA; 20:4 ω-6). Preferably the weight ratio DHA/AA in the present solid-in-oil dispersed composition is at least about 5, preferably at least about 10, more preferably at least about 15, even more preferably at least about 30, and most preferably at least about 60. The present solid-in-oil dispersed composition preferably contains at least one PUFA from a fish oil, algal oil or egg lipid source.

The disclosed formulations are prepared either alone or with an additional therapeutic agent(s) in the form of dosage units for oral administration. The agent(s) can be mixed with optional solid, powdered ingredients, such as, lactose, microcrystalline cellulose, stevia, maltodextrin, saccharose, sorbitol, mannitol, starch, amylopectin, cellulose derivatives, gelatin, or other suitable ingredients, as well as with disintegrating agents and/or lubricating agents such as, magnesium stearate, calcium stearate, sodium stearyl fumarate and polyethylene glycol waxes.

The exact doses of individual agents—polyunsaturated fatty acid (PUFA) and a medium chain fatty acid (MCFA), required to treat (e.g., reduce or prevent) a disorder can be easily discerned by a person skilled in the art, e.g., competent physician, using the patient's history or ascertained from the art.

The disclosed solid-in-oil dispersions comprise a medium chain fatty acid (MCFA) and at least one polyunsaturated fatty acid (PUFA), or an acceptable salt thereof, optionally an acceptable carrier, and a vitamin, an amino acid, a hormone, an element, a nutrient, intermediates of the TCA cycle, Fatty Acid Oxidation and Glycolysis, for preventing or reducing a disorder.

The disclosed solid-in-oil dispersions can comprise vitamins. Non-limiting examples of vitamins include Vitamin A, Vitamin B, Vitamin C, Vitamin D, Vitamin E, Vitamin K, thiamine, riboflavin, niacin, lutein, pantothenic acid, biotin, folic acid, and the like.

The disclosed solid-in-oil dispersions can comprise amino acids. Non-limiting examples include all naturally occurring amino acids irrespective of their configuration, such as, Alanine, Arginine, Aspartic acid, Cysteine (Cystine), Glutamic acid, Glutamine, Glycine, Histidine, Isoleucine, Leucine, Lysine, Methionine, Phenylalanine, L-Phenylalanine, Proline, Serine, Threonine, Trypophan, Tyrosine, Valine and other include Acetyl L-Carinitine Arginate, Alpha-aminoadipic acid, Alpha-amino-N-butyric acid, beta-alanine, beta-amino-isobutyric acid, Carnosine, Citrulline, gamma-amino butyric acid (GABA), hydroxyproline, 1-methylhistidine, 3-methylhistidine, N-Acetyl L-Cysteine, Ornithine amino acid, para-aminobenzoic acid (PABA), Phosphoserine, Phosphoethanolamine, Berberine, Taurine, and the like.

The disclosed grain free solid-in-oil dispersions can comprise hormones. Non-limiting examples include all hormones for human use, such as, thyroid-stimulating hormone (TSH), follicle-stimulating hormone (FSH), luteinizing hormone (LH), prolactin (PRL), growth hormone (GH), adrenocorticotropic hormone (ACTH), vasopressin, oxytocin, thyrotropin-releasing hormone (TRH), gonadotropin-releasing hormone (GnRH), growth hormone-releasing hormone (GHR H), corticotropin-releasing hormone (CRH), somatostatin, dopamine, melatonin, thyroxine (T4), calcitonin, parathyroid hormone (PTH), FGF-23 (phosphatonin), osteocalcin, erythropoietin (EPO), glucocorticoids (e.g., cortisol), mineralocorticoids (e.g., aldosterone), androgens (e.g., testosterone), Adrenalin (epinephrine), norepinephrine, Estrogens (e.g., estradiol), progesterone, human chorionic gonadotropin (HCG), androgens (e.g., testosterone), insulin, glucagon, somatostatin, Amylin, erythropoietin (EPO), Calcitriol, Calciferol, Atrial-natriuretic peptide (ANP), Gastrin, Secretin, Cholecystokinin (CCK), Fibroblast Growth Factor 19 (FGF19), Incretins, Neuropeptide Y, Ghrelin, PYY3-36, Serotonin, Insulin-like growth factor-1 (IGF-1), Angiotensinogen, Thrombopoietin, Hepcidin, Leptin, Retinol Binding Protein 4, Adiponectin, Irisin, and the like.

The disclosed solid-in-oil dispersions can comprise biologically important elements. Non-limiting examples include all elements for human consumption, such as, Sodium, Magnesium, Selenium, Manganese, Chromium, Vanadium, Phosphorus, Sulfur, Tungsten, Arsenic, Boron, Copper, Cobalt, Germanium, Silicon, Nickel, Potassium, Calcium, Iron, Iodine, and the like.

The disclosed solid-in-oil dispersions can comprise biologically important compounds. Non-limiting examples optionally include at least one intermediate of the Citric acid cycle (The citric acid cycle also known as the tri-carboxylic acid cycle (TCA cycle), the Krebs cycle). The intermediate is selected from a group consisting of citric acid, aconitic acid, isocitric acid, α-ketoglutaric acid, succinic acid, fumaric acid, malic acid, oxaloacetic acid, and their nutraceutically acceptable salts and mixtures thereof. The precursor compounds such as 2-keto-4-hydroxypropanol, 2,4-dihydroxybutanol, 2-keto-4-hydroxybutanol, 2,4-dihydroxy-butyric acid, 2-keto-4-hydroxybutyric acid, aspartates as well as mono- and di-alkyl oxaloacetates, pyruvate and glucose-6-phosphate are also included in the definition of intermediates of the Citric Acid Cycle.

In another embodiment, the invention provides a grain free solid-in-oil dispersion comprising a medium chain fatty acid (MCFA) and at least one polyunsaturated fatty acid (PUFA), and at least one ketone body for the combination treatment of a disorder. The preferred ketone body is acetoacetate or β-hydroxy butyrate.

Such combination treatment may be achieved by way of simultaneous, sequential or separate administration of the individual components for the treatment. Such combination methods for treatment employ the disclosed polyunsaturated fatty acid (PUFA), a medium chain fatty acid (MCFA) and at least one solid in oil dispersion, within the dosage range described herein and another active agent within its approved dosage range as described herein.

The disclosed solid-in-oil dispersed composition may further comprise a high concentration of protein. Examples of the protein source that may be used include, but are not limited to, edamame derived protein milk protein isolate, milk protein concentrate, milk protein hydrolysate, soy protein isolate, soy protein concentrate, soy protein hydrolysate, whey protein isolate, whey protein concentrate, whey protein hydrolysate, wheat protein, rice protein, tofu-derived protein, collagen, albumin, gelatin and caseinates. In a preferred embodiment, the protein source is whey protein concentrate, whey protein hydrolysate, or edamame protein isolate. In a preferred embodiment, the protein source is whey protein isolate, or vegetable protein.

The disclosed solid-in-oil dispersed composition may further comprise a high concentration of fat. Examples of the fat sources include animal meat or plant based fats such as nuts. The preferred source of fat is almonds or Brazil nuts.

The following Examples are for illustrating the disclosed grain free solid-in-oil dispersion comprising a medium chain fatty acid (MCFA) and at least one polyunsaturated fatty acid (PUFA), and it not intended to limit the scope of the invention. The experimental examples disclose the preparation and use of a grain free solid-in-oil dispersion comprising a medium chain fatty acid (MCFA), at least one polyunsaturated fatty acid (PUFA) and at least one solid, for providing health benefits and the examples are just intended to be a way of illustrating but not limiting the invention.

### Example 1

### Exemplary Formulation

An exemplary solid-in-oil dispersions is prepared according to standard procedures known it the art. Eicosapentaenoic acid, 2 grams, is mixed with 20 grams of medium chain triglycerides in a suitable container. The temperature is maintained at ≦ about 10° C. during the mixing process. Whey protein, 25 grams, and vitamin D3, 0.0175 g, are added to the homogenized solution. The mixture is stirred until the protein is uniformly dispersed in the oil. The dispersion is stored in cool place maintained at ≦ about 10° C. Following the preparation of solid-in-oil dispersion, an optionally a carrier is added to facilitate the production process.

### Example 2

### Exemplary Formulation

An exemplary solid-in-oil dispersion is prepared according to example 1. Eicosapentaenoic acid, 2 grams, is mixed with coconut powder, 15 grams, in a suitable container. The temperature is maintained at ≦ about 10° C. during the mixing process. Whey protein, 25 grams, and vitamin D3, 0.0175 g, are added to the homogenized solution with stirring until the protein is uniformly dispersed in the oil. The dispersion is stored in cool place maintained at ≦ about 10° C. Following the preparation of solid-in-oil dispersion, an optionally a carrier is added to facilitate the production process.

### Example 3

### Exemplary Formulation

The following formulation is prepared, according to the process disclosed in Examples 1 and 2, using the formula in Table 3.

TABLE 3

| Ingredient | Quantity (g) |
|---|---|
| Docosahexaenoic Acid, | 2 |
| MCTs | 15 |
| Almond flour | 15 |
| Vitamin A | 1000 IU |

### Example 4

### Exemplary Formulation

The following formulation is prepared, according to the process disclosed in Examples 1 and 2, using the formula in Table 4.

TABLE 4

| Ingredient | Quantity (mg) |
|---|---|
| Eicosapentaenoic acid | 2.0 |
| Docosahexaenoic Acid | 1.32 |
| MCT | 20 |
| Hemp Protein | 5 |

### Example 3

### Exemplary Formulation

The following formulation is prepared, according to the process disclosed in Examples 1 and 2, using the formula in Table 3.

TABLE 3

| Ingredient | Quantity (g) |
|---|---|
| Docosahexaenoic Acid, | 2 |
| MCTs | 15 |
| Almond flour | 15 |
| Vitamin A | 1000 IU |

### Example 4

### Exemplary Formulation

Another exemplary formulation is prepared, according to the process described under Example 1, using the formula in table 4.

TABLE 4

| Ingredient | Quantity (mg) |
|---|---|
| Eicosapentaenoic acid | 2.0 |
| Docosahexaenoic Acid | 1.32 |
| MCT | 20 |
| Hemp Protein | 5 |

In addition, 2000 IU of Vitamin D₃ is also added to the composition.

### Example 5

### Exemplary Formulation

The following formulation is prepared, according to the process disclosed in Examples 1 and 2, using the formula in Table 5.

TABLE 5

| Ingredient | Quantity (g) |
|---|---|
| Caprylic triglyceride | 20 |
| Eicosapentaenoic acid (EPA), | 2 |
| α-linolenic acid | 1.0 |
| Hemp Protein | 20 |

### Example 6

### Exemplary Formulation

The following formulation is prepared, according to the process disclosed in Examples 1 and 2, using the formula in Table 6.

TABLE 6

| Ingredient | Quantity g |
|---|---|
| MCTs | 20 |
| Docosahexaenoic Acid (DHA) | 1.5 |
| Eicosapentaenoic Acid | 0.5 |
| L-Carnitine | 1.0 |
| Pea protein | 10.0 |

### Example 7

### Exemplary Formulation

The following formulation is prepared, according to the process disclosed in Examples 1 and 2, using the formula in Table 7.

TABLE 7

| Ingredient | Quantity g |
|---|---|
| Eicosapentaenoic Acid (>90%) | 2.0 |
| Melatonin | 0.5 |
| Rice Protein | 20 |
| Caprylic triglyceride | 20 |

### Example 8

### Exemplary Formulation

The following formulation is prepared, according to the process disclosed in Examples 1 and 2, using the formula in Table 8.

TABLE 8

| Ingredient | Quantity g |
|---|---|
| Eicosapentaenoic Acid | 2.0 |
| Whey Protein | 10.0 |
| Coconut Flour | 10.0 |
| Medium Chain Fatty Acid | 10.0 |

### Example 9

### Exemplary Formulation

The following formulation is prepared, according to the process disclosed in Examples 1 and 2, using the formula in Table 8. The term “PL-DHA” refers to DHA of phospholipids such as lysophosphatidylcholine-DHA, phosphatidylcholine-DHA, phosphatidylinositol-DHA, phosphatidylethanolamine-DHA, sphingomyelin-DHA or mixtures thereof

TABLE 9

| Ingredient | Quantity (g) |
|---|---|
| PL-DHA | 2 |
| Caprylic triglyceride | 20.0 |
| Almond flour | 2.0 |
| Whey Protein | 15.0 |

### Example 10

### Exemplary Formulation

The following formulation is prepared, according to the process disclosed in Examples 1 and 2, using the formula in Table 10.

TABLE 10

| Ingredient | Quantity (g) |
|---|---|
| EPA | 2 |
| MCTs | 20 |
| Vitamin D | 50,000 IU |
| Whey Protein | 25 |
| Banana Dry | 3.0 |

### Example 11

### Exemplary Formulation

The following formulation is prepared, according to the process disclosed in Examples 1 and 2, using the formula in Table 11.

TABLE 11

| Ingredient | Quantity (g) |
|---|---|
| DPA | 2 |
| Caprylic triglyceride | 20 |
| L-Carnitine | 1.0 |
| Whey Protein | 15.0 |

### Example 12

### Exemplary Use in a Breakfast Composition

A grain free nutraceutical composition is prepared according to the procedures disclosed above. Eicosapentaenoic acid, 2 grams, and coconut powder, 20 grams, are mixed with in a suitable container. The temperature is maintained at ≦ about 10° C. during the mixing process. Whey protein, 25 grams, and vitamin D3, 0.0175 g, are added to the homogenized solution with stirring until the protein is uniformly dispersed in the oil. Following the preparation of the health food product, an optionally a carrier is added to facilitate the production process. The solid-in oil composition is stored in a cool place maintained at less than about 10° C. until further use.

A standard serving (44 grams) of breakfast cereal (for example, Raisin Bran) is placed in a container and one serving of (48 grams) of MCFA+PUFA+Whey Protein composition granules is added and mixed at room temperature. The health food product prepared using the grain free nutraceutical composition according to this procedure is stored at room temperature till consumption.

### Example 13

### Exemplary Yogurt Dessert

A standard yogurt dessert is prepared as follows: Eicosapentaenoic acid, 1.5 grams, and docosahexaenoic acid, 0.5 grams, are mixed with coconut powder, 20 grams, in a mixer. The temperature is maintained at less than about 10° C. during the mixing process. Whey protein, 25 grams, and vitamin D3, 0.0175 g, are added to the homogenized solution with stirring until the protein is uniformly dispersed in the MCFA/PUFA/Protein composition. The composition is mixed with standard plain yogurt and flavorings, 20 g. Preservatives and additives are added and mixed as needed and stored aside under refrigeration until consumed. The key requirement is the proportion of at least one medium chain fatty acid (MCFA) and protein provides at least 50% total calories of one serving of the consumed food product prepared according to this procedure.

### Example 14

### Nutritional Drink

Nutritional drinks are prepared from food prepared by standard methods. Eicosapentaenoic acid, 1.5 grams, and docosahexaenoic acid, 0.5 grams, are mixed with MCT oil or powder, 20 grams, in a mixer. The temperature is maintained at less than about 10° C. during the mixing process. Whey protein, 25 grams, and vitamin D3, 0.0175 g, are added to the homogenized solution with stirring until the protein is uniformly dispersed in the composition. The composition is added to a mixture of daily vitamins at recommended daily levels, 5 grams cocoa, additives and preservatives. The key requirement is the proportion of the MCFA and Protein provide at least 50% of one serving of the nutritional drink prepared according to this procedure.

Additional energy drink formulations such as a ready to drink beverage, powdered suspension, nutritional Bars, and the like can be prepared, by those skilled in the art, using at least one MCFA, at least one PUFA and at least one protein, disclosed herein.

### Example 15

### Powdered Beverage

A powdered beverage can be formed as described below: Eicosapentaenoic acid, 2.0 grams, and MCFA, 20 grams, are mixed, in a mixer. The temperature is maintained at less than about 10° C. during the mixing process. Whey protein, 25 grams, and vitamin D3, 0.0175 g, are added to the homogenized solution with stirring until the protein is uniformly dispersed in the composition. The composition is added to L-carnitine 250-500 mg, sucralose 8-15 g, and flavorings 0-1 g, as needed to facilitate the production of a beverage.

### Example 16

### Energy Bar

An energy bar having ˜10-30 grams of MCFA, ˜2-3 grams of at least one PUFA, ˜15-50 grams of at least one protein, L-carnitine ˜250-500 mg, glycerin ˜1-5 g, dry banana powder ˜5-10 grams, cocoa 2-7 g, coating ˜15-25 g is prepared. Eicosapentaenoic acid, 2 grams, and coconut powder, 20 grams, are mixed with in a suitable container. The temperature is maintained at ≦ about 10° C. during the mixing process. Whey protein, 25 grams, and vitamin D3, 0.0175 g, are added to the homogenized solution with stirring until the protein is uniformly dispersed in the oily composition. Following the preparation of the health food product, other ingredients, e.g., L-carnitine, from about 250 to about 500 mg, glycerin, from about 1 to about 5 g, dry banana powder from about 5 to about 25 g, cocoa from about 2 to about 7 g, and a coating from about 15 to about 25 g are used as needed to facilitate the production of energy bar. The food product was stored in a cool place maintained at less than 20° C. until further use.

### Example 17

### Burger Patties

A burger patty health food product having ˜10-30 grams of MCFA, ˜2-3 grams of at least one PUFA, ˜15-50 grams of at least one protein, L-carnitine ˜250-500 mg, glycerin ˜1-5 g, corn syrup solids ˜5-25 g, salts and additives. Eicosapentaenoic acid, 2 grams, is mixed with coconut powder, 20 grams, in a mixer. The temperature is maintained at less than about 10° C. during the mixing process. Whey protein, ˜15-50 grams, and vitamin D3, 0.0175 g, are added to the homogenized solution with stirring until the protein is uniformly dispersed in the oily composition. Following the preparation of the grain free nutraceutical composition, other ingredients, e.g., L-carnitine from about 250 to about 500 mg, glycerin from about 1 to about 5 g, natural gum from about 5 to about 25 g, and a suitable amount of salt and additives are used as needed to facilitate the production of burger patty. The food product was stored in a cool place maintained at less than 20° C. until consumption as a standard meal.

### Example 18

### Pasta

A pasta health food product having ˜10-30 grams of MCFA, 2-3 grams of at least one PUFA, 15-50 grams of at least one protein, L-carnitine 250-500 mg, glycerin 1-5 g, corn syrup solids 5-25 g, salts and additives is prepared. Eicosapentaenoic acid, 2 grams, and is mixed with coconut powder, 20 grams, in a mixer. The temperature is maintained at less than about 10° C. during the mixing process. Whey protein, 15-50 grams, and vitamin D3, 0.0175 g, are added to the homogenized solution with stirring until the protein is uniformly dispersed in the oily composition. Following the preparation of grain free nutraceutical composition, other ingredients, e.g., L-carnitine, from about 250 to about 500 mg, a suitable amount of salt and additives are used as needed to facilitate the production. The food product was stored in a cool place maintained at less than 20° C. until consumption as a standard meal optionally with a pasta sauce.

### Example 19

### Salad

A salad health food product having suitable quantities of salad vegetables, ˜10-30 grams of MCFA, 2-3 grams of at least one PUFA, 15-50 grams of at least one protein, L-carnitine 250-500 mg, glycerin 1-5 g, sucralose 5-25 g, salts and additives is prepared. Eicosapentaenoic acid, 2 grams, is mixed with coconut powder, 20 grams, in a mixer. The temperature is maintained at less than about 10° C. during the mixing process. Whey protein, 15-50 grams, and vitamin D3, 0.0175 g, are added to the homogenized solution with stirring until the protein is uniformly dispersed in the oily composition. Following the preparation of grain free nutraceutical composition, other ingredients, e.g., L-carnitine from about 250 to about 500 mg, a suitable amount of salt and additives are used as needed to facilitate the production. The food product was added to suitable quantity of salad vegetables and consumed as a standard salad optionally with a salad dressing.

### Exemplary Formulations of a Health Food Product

The method of preparing a standard grain free nutraceutical composition is outlined above, the composition of MCFA, PUFA, protein content and other dietary agents fortified may vary as exemplified (examples 20-24) below in Table 12.

TABLE 12

| Ingredient | Ex. 10 | Ex. 11 | Ex. 12 | Ex. 13 | Ex. 14 |
|---|---|---|---|---|---|
| Vitamin D | 1000 IU | 1000 IU | 2000 IU | 1000 IU | 5000 IU |
| Docosahexaenoic Acid, | 2 | 0 | 1.32 | 2 | 2 |
| Eicosapentaenoic Acid | 0 | 2 | 2.1 | 0 | 0 |
| MCFAs | 15 | 20 | 20 | 20 | 5 |
| Almond flour | 15 | 0 | 0 | 0 | 0 |
| Soy Protein | 0 | 20 | 0 | 0 | 0 |
| Rice Protein | 0 | 0 | 0 | 0 | 5 |
| Whey Protein | 0 | 0 | 20 | 0 | 0 |
| Melatonin | 0 | 0 | 0 | 0.5 | 0 |
| Hemp Protein | 0 | 0 | 0 | 20 | 5 |
| L-Carntine | 1 | 1 | 0 | 0 | 1 |

### Pharmacological Studies

While not wishing to be bound by theory, it is submitted that the polyunsaturated fatty acids work by acting at different sites and aspects of a disease. This modulation is likely influenced by the relative ratios of polyunsaturated fatty acids. PUFAs are known to increase anti-inflammatory properties, enhance the beta-oxidation in peroxisomes and improve the integrity of cell membranes. Similarly, upon administration of a medium chain fatty acid (MCFA), which is known to undergo β-oxidation in hepataocytes, generate supply ketone bodies and release them to circulation. The ketone bodies can be utilized by extra-hepatocytic cells such as neuron, retinal cells, heart, nephron, and other affected cells, or excreted from the mammal. The inventive solid-in-oil dispersion combines the therapeutic properties of a polyunsaturated fatty acid and the properties of a medium chain fatty acid, such as anti-inflammatory properties, energy homeostasis, or membrane properties. In addition, the disclosed dispersion can be easily incorporated into å supplement or a food facilitating patient compliance. Thus, the changes expected in the levels of IL-6, TNF, and/or ketone bodies upon administering either the disclosed solid-in-oil dispersion directly or the foods prepared using the disclosed solid-in-oil dispersion can provide health improvement. Increase in IL-6, TNF and Ketone bodies are reported to improve clinical symptoms associated with obesity, diabetes, rheumatoid arthritis (RA), schizophrenia, diabetic retinopathy and nonalcoholic fatty liver diseases (NALFD).

### Example 25

### Lipid Panel, Interleukin-6, TNF, Vitamin D and Ketone Bodies Studies

Primary Objectives:

A study to evaluate the effect of a grain free solid-in-oil dispersion on the lipid panel, serum concentrations of IL-6, TNF, vitamin D and Ketone bodies in healthy volunteers by exploring changes in the lipid a, serum concentration of IL-6, TNF, and Ketone bodies in healthy volunteers.

Study Design:

An open label study of the effects of solid-in-oil dispersions on the lipid panel and on the plasma concentration of ketone bodies, Interleukin-6, Vitamin D₃ and Tumor Necrosis Factor.

The study was divided into two periods; a) a baseline period of three days and b) a treatment period of seven days, with solid-in-oil dispersion. During the baseline period, the subjects underwent a baseline characterization phase where the baseline lipid panel, IL-6. TNF and ketone bodies were determined. The during the treatment period, the subjects were administered a grain free solid-in-oil dispersion prepared as disclosed Example 4 for seven days. Upon completion of treatment period, lipid panel, serum ketone bodies, 25 hydroxy vitamin D, IL-6 and TNF values were determined on day 8.

### Study Arms and Medications

Treatment Arm (Solid-In-Oil Dispersion Arm):

Example 4 consisting of 20 grams of MCT, 2 grams of EPA, 1.32 grams of DHA, 2000 IU of Vitamin D₃, and 5 grams of Hemp Protein.

Inclusion Criteria:

Prior to randomization, subjects eligible for enrollment in the study met all of the following criteria:
Healthy Volunteers aged 18 years or older.
A female subject is eligible to enter and participate in the study if she:
   Is of non-childbearing potential or
   Is of child-bearing potential, is not lactating and has a negative pregnancy test ≦7 days prior to study treatment initiation and agrees to use one of the sponsor specified highly effective methods for avoiding pregnancy.
A documented diagnosis of good health.

Exclusion Criteria:

Subjects having any of the following conditions were excluded from the study:
A history of previous ketoacidosis;
Substance-induced psychotic disorder;
Acute suicidal or aggressive behavior;
Neurological disorders (e.g., epilepsy);
Any structural brain changes apparent on clinical examination;
Medical condition or disorder that would interfere with the action, absorption, distribution, metabolism, or excretion of ω-3 PUFA or a medium chain fatty acid (MCFA), or, in the investigator's judgment:
Is suffering from a condition considered to be clinically significant and could pose a safety concern, could interfere with the accurate assessment of safety or efficacy, or, could potentially affect a subject's safety or study outcome.

If the subject had taken ω-3 Polyunsaturated Fatty Acids (PUFA), MUFA supplements within one week of being included in the trial, he/she must undergo a three days of washout period to remove the supplement from their system.

Investigational Agents:

The active ingredients in the investigational product—are Medium Chain Triglycerides, Eicosapentaenoic Acid (EPA) and Docosahexaenoic Acid (DHA). All three active agents have been extensively studied in humans.

MCTs:

Medium chain triglycerides (MCTs) are a class of lipids in which three saturated fats are bound to a glycerol backbone. MCTs are distinguished from other triglycerides in that each fat molecule is between six and twelve carbons in length. The specific composition of MCTs used in Example 4 are below:
Caproic Acid Triglyceride (C₂₄H₄₄O₆) with C₆ hydrocarbon chains; Not more than 6%;
Caprylic Acid Triglyceride (C₂₇H₅₀O₆) with C₈ hydrocarbon chains: Between 55% to 85% of the composition. Generic name-Caprylic triglyceride (Other names: Tricaprylin; Octanoin, tri-; Caprylic acid triglyceride; Caprylin; Glycerol trioctanoate; Glyceryl trioctanoate; Octanoic acid triglyceride), Chemical name: 2,3-bis(octanoyloxy)propyl octanoate, Molecular formula: C₂₇H₅₀O₆, Molecular weight: 470.68 and Melting point: 8-10° C.;
Capric Acid Triglyceride (C₂₉H₅₄O₆) with C10 hydrocarbon chains: Between 15% to 40% of the composition. Generic name: Capric Triglyceride, Chemical Name-2,3bis(decanoyloxy)propyl decanoate, Molecular Formula—C₂₉H₅₄O₆, Molecular Weight—425.7 and Melting point: 12-14° C.;
Lauric Acid Triglyceride (C₃₂H₆₀O₆) with C₁₋₂ hydrocarbon chains: Not more than 4%;
Eicosapentaenoic acid (EPA): EPA is a long chain, 20 carbon, omega-3 polyunsaturated fatty acid (PUFA) found in the diet such as in fish oil. Upon consumption, it undergoes demethylation of ethyl-EPA to EPA. Enterocyte uptake is followed by re-esterification, chylomicron formation and secretion into the lymph before systemic distribution. Molecular formula: C₂₀H₃₀O₂, Molar Weight: 302.45 g/mol and Melting point: Oil with a melting point of 54° C.;
Docosahexaenoic Acid (DHA): SHA is long chain 22-carbon atom omega-3-polyunsaturated fatty acid (PUFA) found in fish oil. Molecular formula: C₂₂H₃₂O₂, Molar Weight: 328.488 g/mol, Melting point: Oil with a melting point of −44° C.

Pharmacology:

Docosahexaenoic acid (DHA) has been extensively studied in humans and animals in combination with EPA. DHA is recognized as being essential for normal development of the brain and retina during foetal development and the first two years of life. The European Food Safety Authority (EFSA) stated that the consumption of n-DHA has not been associated with adverse effects in healthy children or adults at observed intake levels. Therefore, supplemental combined intakes of EPA and DHA at doses of up to 5 g/day, and supplemental intakes of EPA alone up to 1.8 g/day, do not raise safety concerns for adults. Supplemental intakes of DHA alone up to about 1 g/day do not raise safety concerns for the general population.

Statistical Methods: SAS software was used for analysis performed by using the analysis of variance. The results were processed using repeated measures analysis of variance and were considered significant when P<0.05

Subject Population(s) for Analysis:

The all subjects were used for all study analyses. For the purposes of this study, the all-randomized population is defined as any subject randomized into the study, regardless of whether they receive study drug.

### Results

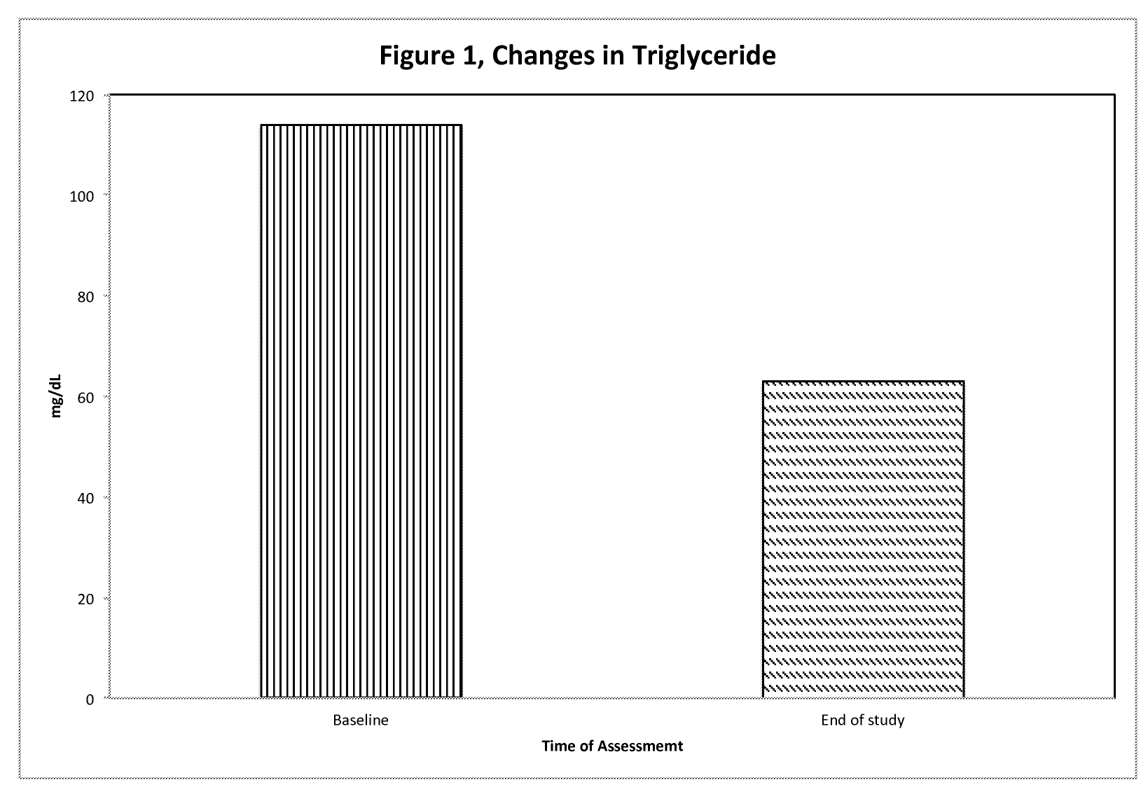

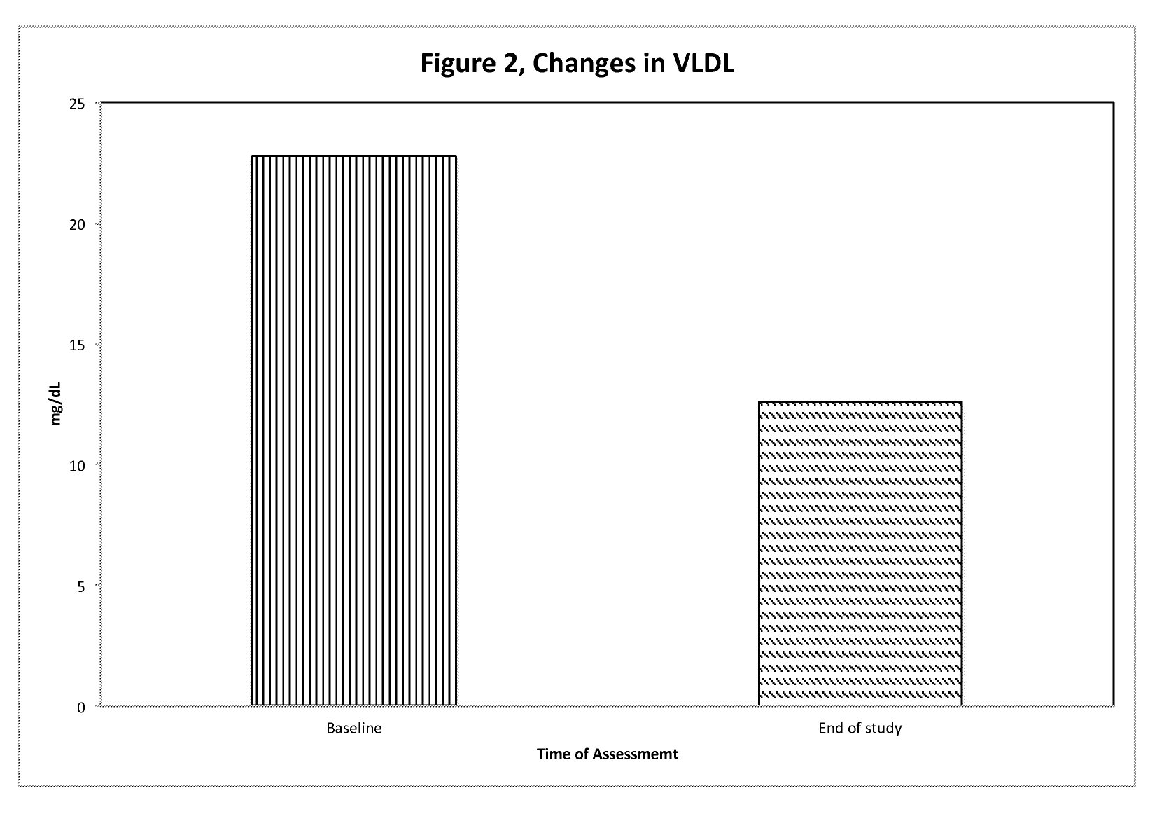

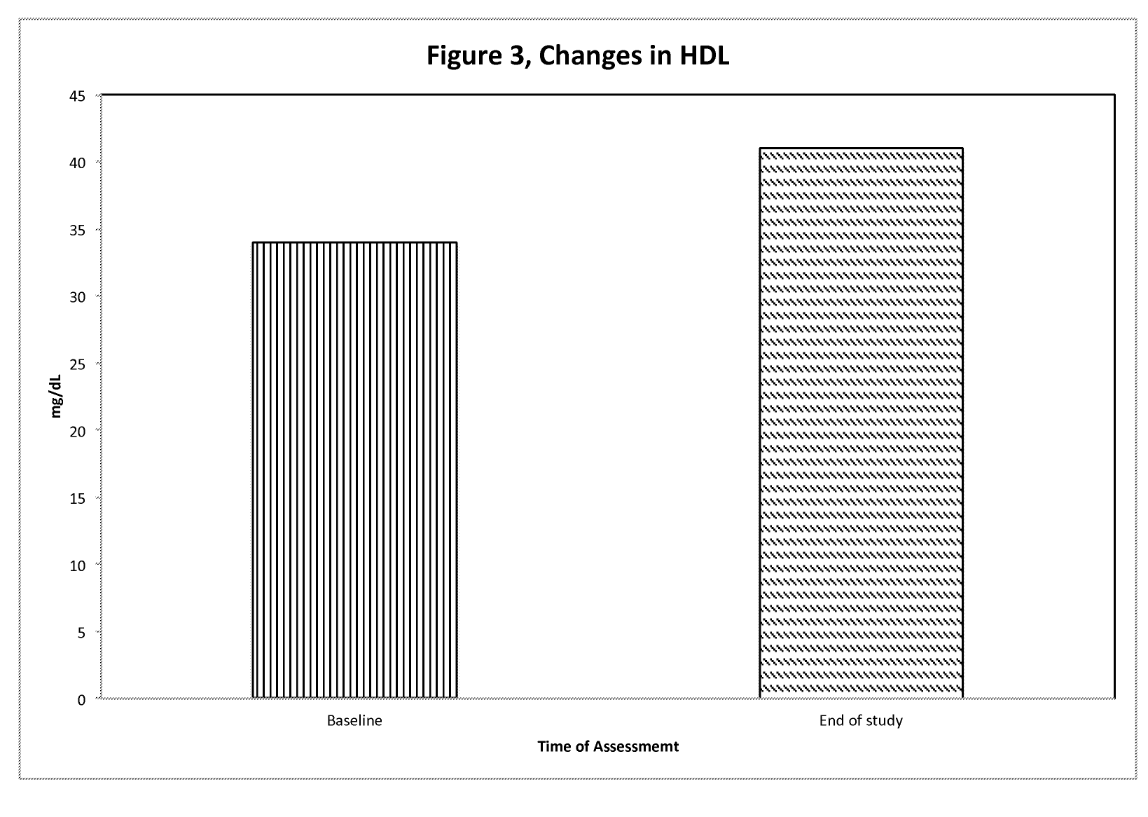

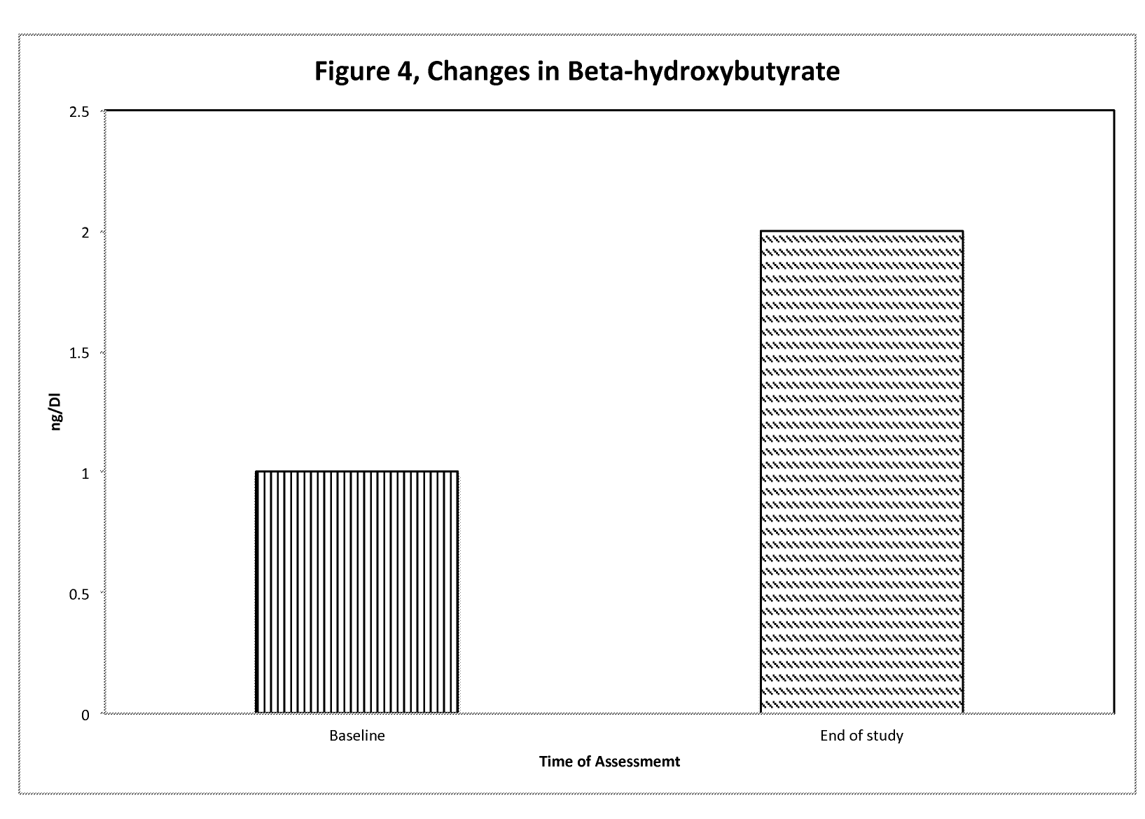

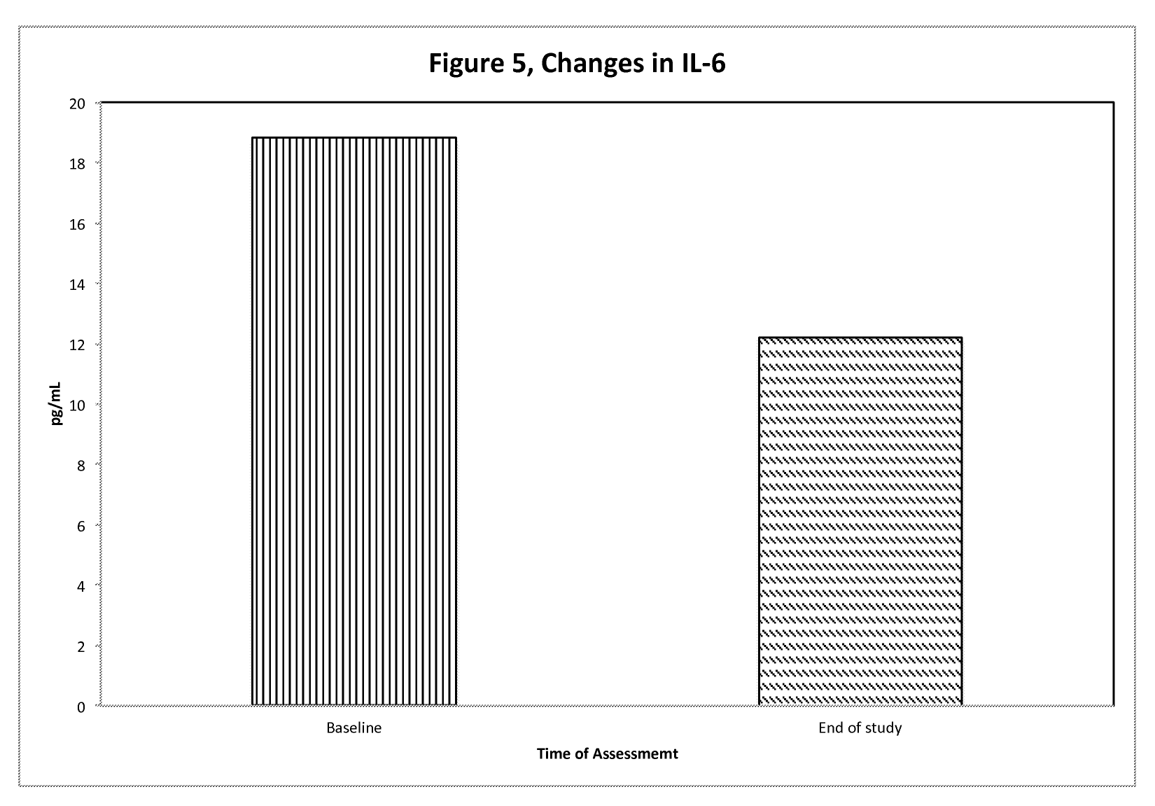

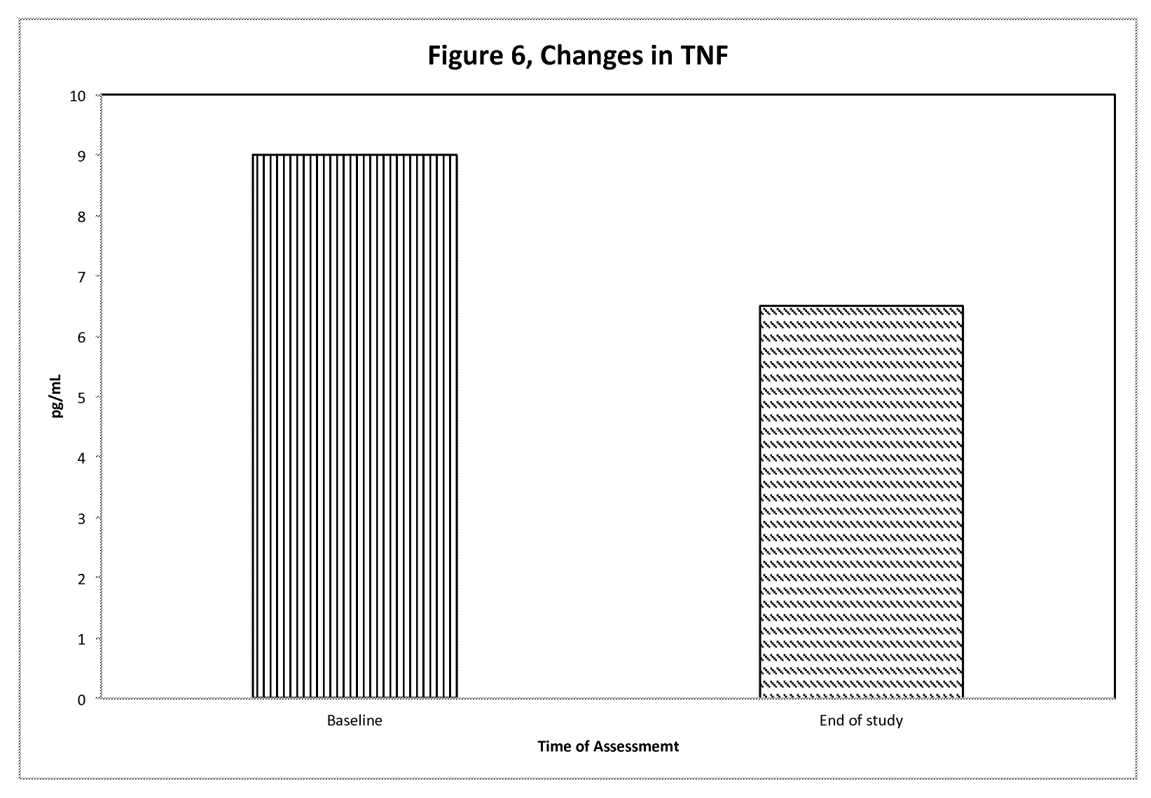

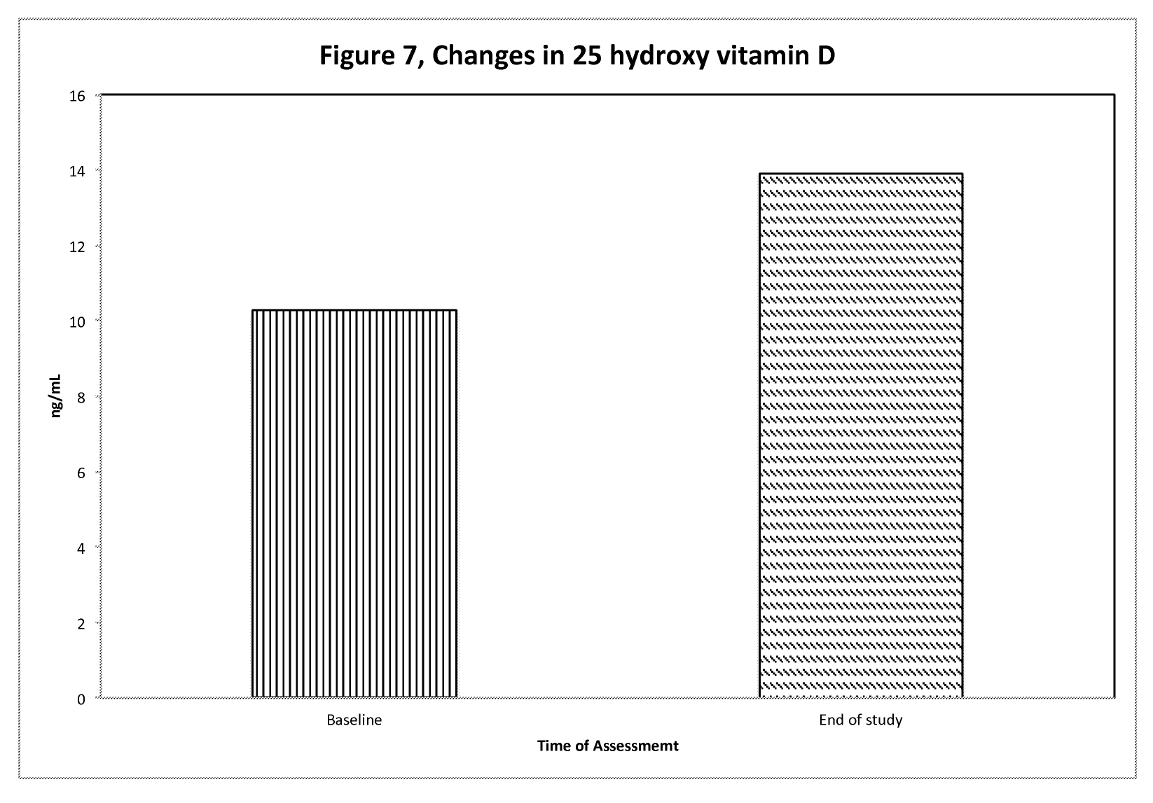

FIG. 1 illustrates the change in serum triglyceride concentration from baseline to end of study.
FIG. 2 illustrates the change in serum VLDL concentration from baseline to end of study.
FIG. 3 illustrates the change in serum HDL concentration from baseline to end of study.
FIG. 4 illustrates the change in the mean serum concentrations of β-hydroxy butyrate during the study.
FIG. 5 illustrates the change in the mean serum concentrations of IL-6 during the study.
FIG. 6 illustrates the change in the mean serum concentrations of TNF during the study.
FIG. 7 illustrates the changes in serum concentration of 25 hydroxyl vitamin D during the study.

### Other Health Related Uses

Without being bound to any specific theory, the root cause for the exacerbation of most of the modern diseases like cancer, obesity, diabetes, fatty liver & inflammatory bowel diseases and for the increased prevalence of certain genetic diseases like primary biliary cirrhosis, autism, ADHD is believed to be the modern diet. The western diet is relatively devoid of MCTs and very high in pro-inflammatory Omega-6-Polyunsaturated fatty acids. Fasting and high level of physical activity which were very common during the era when modern human genes were evolved several hundred years ago are either very uncommon or inadequate because social and technological changes. Thus, the disclosed grain free solid-in-oil dispersion and food products developed address this unmet need thereby useful for a number of diseases.

Ketone bodies are also naturally formed in the liver under conditions of prolonged fasting. Since fasting is uncommon in most developed countries and modern Western diets are relatively void of MCTs, these alternative food products provide energy to cells that do not occur naturally for most people. Dietary sources rich in MCTs include whole-fat dairy products, palm kernel oil and coconut oil. MCTs are transported directly to the liver, where they are metabolized into ketone bodies. Similarly, the presence of high dosage of omega-3-PUFAs in the food products, help improve the anti-inflammatory profile of the cells, enhance the peroxisomal-β-oxidation and improve the membrane integrity. When taken together with carefully chosen protein, flour and isolates, MCTs and PUFAs improve the overall energy homeostasis and immunity thereby providing health benefits against a number of modern diseases as described below.

Multiple Sclerosis (MS):

Multiple sclerosis (MS) is a disease of the central nervous system. The exact cause of Multiple Sclerosis in humans has not been determined. Multiple sclerosis (MS) is a complex disease of a heterogeneous nature. Its etiology has caused much controversy, and still remains unknown in the medical community after decades of research. The first description of MS as a neurological condition that afflicts the myelin sheath insulating long extensions of the axon conducting electrical signals from one neuron to another. MS is identified as a disease of young adulthood and does exist worldwide.

There is no known cure for MS. There are partially effective strategies are available to modify the disease course, treat exacerbations, attacks, relapses, or flare-ups, manage symptoms, improve function and safety, and provide emotional support. The treatment options for multiple sclerosis include the use of disease modifying agents with interferon beta, such as, Avonex (interferon beta-1a), Betaseron (interferon beta-1b), Rebif (interferon beta-1a), Extavia (interferon beta-1b) and others, such as, Copaxone (glatiramer acetate), Gilenya (fingolimod), Novantrone (mitoxantrone), and even a monoclonal antibody, such as, Tysabri (natalizumab). However, these treatments do not fully enhance the quality of life for people living with MS. There is recent research that reports strong evidence that axonal degeneration is a critical factor in the etiology of MS and have dysfunctional complexes I, III and IV because of electron transport system. In addition, MS is characterized by vitamin D deficiency and inflammation indicating the mitochondria function is crucial in preserving axonal integrity in both acute inflammatory and progressive stages of the disease. Since the disclosed composition comprises MCTs, PUFA, and Vitamin D, it can be helpful in producing ketone bodies that can potentially inhibit free radical production, supply anti-inflammatory eicosanoids and docosanoids and nutritional support.

Epilepsy:

Clinical use of ketones to treat CNS disorders has been ongoing for decades in the form of the ketogenic diet. Typically, ketogenic diets are rich in fat and low in carbohydrates and proteins. Unfortunately, this combination reduces palatability. A ketogenic diet induces a decrease in blood sugar levels and an increase in ketone bodies via conversion of fatty acids in the liver. A ketogenic diet has shown to be effective in pharmaco-resistant forms of epilepsy, including catastrophic cases of infantile spasms, the multiple seizure types associated with the Lennox-Gastaut syndrome, and certain inherited metabolic disorders. Ketogenic diets increase levels of circulating ketone bodies in the blood and have been shown to reduce seizures by more than 50%.

While a typical ketogenic diet consists of about 88% fat, about 10% protein, and about 2% carbohydrates and is a valuable adjunct in the management of epilepsy in children and adults with seizure disorder, palatability and patient compliance are major issues. Studies have shown that 53.9% of patients had a more than 75% reduction in seizure frequency 1 month and recent studies have shown that children who remained on the ketogenic diet for more than 1 year, and who had a good response to the diet, had positive outcomes at 3-year and 6-year follow ups, after initiation of the diet provided the compliance is good.

By using MCTs and PUFAs in a grain free solid-in-oil dispersion with an edible solid material, the disclosed composition promotes formation of ketone bodies, improves anti-inflammatory effects and palatability considerably. Thus the disclosed dietary supplements and food products are especially useful as standard nutritional support under the supervision of a physician in the dietary management of epilepsy.

Ophthalmic Disorders:

Systemic oxidative stress, dysfunctional cellular processes and malfunctioning mitochondria are implicated in the pathogenesis of diseases such as retinopathy, AMD, cataract, glaucoma and retinitis pigmentosa. It is reported that the pathogenesis of glaucoma, AMD and Alzheimer's has common pathways. The brain, which comprises only about 2% of the body weight, is one of the highest energy demanding tissues of the human body and consumes 20% of the total oxygen and about a quarter of the total glucose used for energy supply. Within the brain, the visual system reportedly ranks amongst the highest energy-consuming systems. Thus, the visual system requires high performing energy supply system. In ophthalmic diseases, the primary defect often affects glucose metabolism, which is crucial for energy supply. For example, in proliferative diabetic retinopathy, the blood supply to the retina is reportedly reduced. Because blood transports oxygen and glucose, it is proposed the energy supply is compromised in diabetic retinopathy.

The disclosed compositions, in combination with specific agents, such as, vitamin A, Luetin, alpha-ketoglutarate, provide unique products for large market indications such as diabetic retinopathy, age related macular degeneration, glaucoma in addition to smaller underserved markets like retinitis pigmentosa, and the like.

Psychiatric Disorders:

Unlike other organs, the brain, which is energy intensive, can only consume glucose and ketone bodies for their metabolic processes. It is being increasingly reported that disturbed energetic metabolism and/or reactive oxygen species production take part in the pathophysiology of psychiatric disorders and more specifically in schizophrenia, bipolar disorder and major depressive disorder. The TCA cycle plays a central role in the oxidation of all substrates and the function of the ATP producing oxidative phosphorylation machinery in neurons. Oxidative phosphorylation is reportedly involved in synaptic signaling and plays a role in ion homeostasis in presynaptic nerve terminals. Proteome analysis of schizophrenic patient reports eleven down-regulated and fourteen up-regulated proteins, most of them related to energy metabolism. Metabolite marker studies in schizophrenic patients strongly support the role of energy metabolism dysfunction particularly glycolysis. Similarly, recent research indicates the central role of energy metabolism, more specifically oxidative phosphorylation, and mitochondrial distress in major depressive disorders and bipolar disorders.

The disclosed compositions can generate ketone bodies for energy deficient neurons, providing w-omega PUFA for addressing lipid, neuro-protective, and anti-oxidant deficiencies in different psychiatric disorders such as schizophrenia, MDD, bipolar disorder, and the like. In addition, clinical studies indicate long-chain ω-3 PUFAs reduce the risk of progression to a psychotic disorder and may offer a safe and efficacious strategy for indicated prevention in young people with sub-threshold psychotic states. The formulation also synergistically improves β-oxidation of fatty acids, up-regulates anti-inflammatory cytokine IL-10, and down-regulates pro-inflammatory cytokines.

Gastrointestinal and Liver Diseases:

Typically, lipid and glucose metabolism are in constant equilibrium in liver. If either of them is dysfunctional, it can result in many gastrointestinal and liver diseases such as celiac disease; Whipple disease, Crohn's disease; enteritis; gluten enteropathy; intestinal lymphangiectasia; chylous ascites; chylothorax; fistulas, cholestasis, stomach or duodenum; biliary atresia; obstructive jaundice; primary biliary cirrhosis; blind loop syndrome; gastrointestinal cancer; pancreatitis; cystic fibrosis where lipid breakdown or lipid uptake is compromised, in situations in which gall bladder or pancreas is dysfunctional, or anomalies occur in the lymph flow.

It has long been recognized that liver diseases like alcoholic and nonalcoholic fatty liver diseases, primary biliary cirrhosis, chronic liver failure, nonalcoholic steatohepatitis (NASH), have dysfunctional energy metabolism characterized by increased fatty acid oxidation. There are publications that report a link between fatty liver disease and mitochondrial dysfunction, inflammation and dysfunctional mitochondrial and peroxisomal β-oxidation of fatty acid against their ω-oxidation in endoplasmic reticulum (ER). The disruption of equilibrium among gluconeogenesis, TCA cycle, fatty acid oxidation and oxidative phosphorylation and the signaling mechanisms lead to fatty liver diseases.

The disclosed compositions of MCT dispersion with large doses of eicosapentaenoic acid and docosahexaenoic acid are especially useful. MCTs are completely broken down into fatty acids by pancreatic enzymes and, unlike LCTs, MCTs can even be taken up in the absence of bile acids or pancreatic enzymes. Medium chain fatty acids (MCFAs) are delivered directly to the blood, where they are transported in a complex with serum albumin. Therefore MCTs do not induce lymph flow. In summary, MCTs are taken up more quickly, more directly and more completely in the circulation than LCTs. MCTs and PUFAs prevent triglyceride accumulation and enhance (ω-3 polyunsaturated acids) peroxisomal β-oxidation. In addition, the formulation also synergistically improves fatty acid oxidation, up-regulates anti-inflammatory cytokine IL-10 and down regulates pro-inflammatory cytokines. For these reasons, solid-in-oil dispersions disclosed herein can be used in the dietary management of gastrointestinal and liver diseases.

Inflammatory Disorders:

While the interplay between the nervous, endocrine, and immune systems is recognized to be involved in the pathophysiology of chronic inflammatory diseases (CIDs), the recent findings highlight the role of adipose and energy regulation in inflammatory diseases. Inflammatory disorders such as irritable bowel syndrome (IBS), inflammatory bowel diseases (IBD), rheumatoid arthritis, etc. are characterized by higher level of pro-inflammatory cytokines. For example, irritable bowel syndrome (IBS) is reportedly characterized by an augmented cellular immune response with enhanced production of pro-inflammatory cytokines such as IL-6, TNF-α as reported in a study of 55 IBS patients as compared to age-sex matched healthy controls. Similarly, most of the effective monoclonal antibodies approved to manage rheumatoid arthritis, e g, inhibit IL-6 and TNF-α cytokines13 improve the disease profile. A number of studies indicate that energy regulatory dysfunction/oxidative stress as a consequence of increased production of ROS and reactive nitrogen species (RNS) is important in the pathogenesis of IBD. The disclosed dispersion of MCTs, PUFAs and edible solid food can improve fatty acid oxidation, may up-regulate anti-inflammatory cytokine IL-10, and down-regulate pro-inflammatory cytokines. In addition, MCTs generate ketone bodies, enhance anti-oxidant/ROS scavenging capabilities, and are useful in addressing energy and metabolic/lipid deficiencies in inflammatory disorders. The ketone bodies generate conditions similar to what prevails under fasting or under a low carbohydrate diet that is reported to significantly reduce symptoms of IBS.

The abbreviations used herein have their conventional meaning within the chemical and biological arts. The disclosures of each and every patent, patent application, and publication cited herein are expressly incorporated herein by reference in their entirety into this disclosure. In the case of any inconsistencies, the present disclosure, including any definitions therein will prevail. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure and the claims shown below are not limited to the illustrative embodiments set forth herein.

### BIBLIOGRAPHY

1. Malcom Peet et al., A Dose-Ranging Study of the Effects of Ethyl-Eicosapentaenoate in Patients With Ongoing Depression, Arch. Gen. Psychiatry 2003, 59, 913-919.
2. Dominique Lemaitre-Delaunay, et al., Blood compartmental metabolism of docosahexaenoic acid (DHA) in humans after ingestion of a single dose of [¹³C]DHA in phosphatidylcholine, J. Lipid Res. 1999. 40: 1867-1874.
3. Paval Flashs et al., Cellular and molecular effects of n-3 polyunsaturated fatty acids on adipose tissue biology and metabolism, Clinical Science (2009) 116.
4. Greg Cole et al., DHA May Prevent Age-Related Dementia, J. Nutr. 140: 869-874, 2010.
5. M Picq et al., DHA Metabolism: Targeting the Brain and Lipoxygenation, Mol Neurobiol (2010) 42:48-51.
6. Patricia Wineright, Dietary essential fatty acids and brain function-a developmental perspectives on mechanisms, PNAS, (2002), 61, 61-69.
7. Frazer Russell, Distinguishing Health Benefits of Eicosapentaenoic and Docosahexaenoic Acids, Mar. Drugs 2012, 10, 2535-2559.
8. Ken D Stark, Docosahexaenoic acid and n-6 docosapentaenoic acid supplementation alter rat skeletal muscle fatty acid solid-in-oil dispersed composition, Lipids in Health and Disease 2007, 6:13.
9. V Nobili, et al., Docosahexaenoic acid for the treatment of fatty liver: Randomised controlled trial in children, Nutrition, Metabolism & Cardiovascular Diseases (2012) xx, 1e5.
10. Sophia Frangou, Randomised double-blind placebo-controlled study; Efficacy of ethyl-eicosapentaenoic acid in bipolar depression, BJP 2006, 188:46-50.
11. Jeremy Van Raamsdonk et al., Ethyl-EPA treatment improves motor dysfunction, but not neurodegeneration in the YAC128 mouse model of Huntington disease, Experimental Neurology Volume 196, Issue 2, December 2005, Pages 266-272.
12. A. P Simpoulous, Evolutionary aspects of diet, the omega-6/omega-3 ratio and genetic variation: nutritional implications for chronic diseases, Biomedicine & Pharmacotherapy 60 (2006) 502-507.
13. Timothy Trebble, Inhibition of tumour necrosis factor-a and interleukin 6 production by mononuclear cells following dietary fish-oil supplementation in healthy men and response to antioxidant co-supplementation, British Journal of Nutrition (2003), 90, 405-412.
14. Jonathon Buckle, et al., Long-Chain Omega-3 Polyunsaturated Fatty Acids May Be Beneficial for Reducing Obesity—A Review Nutrients 2010, 2, 1212-1230.
15. Brian Ross et al., Omega 3-Faty Acid Which one for which disorder—Which Disorder and Which Fatty Acid, Lipids in Health and Disease 2007, 6:21.
16. Elizabeth Sublette, Meta-analysis of the effects of Eicosapentaenoic Acid in clinical trials in depression, J. Clin. Psychiatry, 2007, el.
17. George Booz et al., Use of Metabolomic Profiling in the Study of Arachidonic Acid Metabolism in Cardiovascular Disease, Congestive Heart Failure (2011) Volume 17, Issue 1, pages 42-46.
18. Simon H S Pearce et al., Diagnosis of and management of vitamin Deficiency, BMJ 2010; 340: b5664.
19. Robert Simpson, Selective Knockout of the Vitamin D Receptor in the Heart Results in Cardiac Hypertrophy Is the Heart a Drugable Target for Vitamin D Receptor Agonists? Circulation. 2011; 124: 1808-1810.
20. Danny Bruce, Vitamin D and host resistance to infection? Putting the cart in front of the horse, Experimental Biology and Medicine 2010, 235:921-927.
21. Armin Zittermann et al., Vitamin D deficiency and mortality risk in the general population: a meta-analysis of prospective cohort studies, Am J Clin Nutr 2012; 95:91-100.
22. Karin Zwerina, et al., Vitamin D receptor regulates TNF-mediated arthritis, Ann Rheum Dis 2011; 70:1122-1129.
23. Armin Zittermann et al., Vitamin D supplementation enhances the beneficial effects of weight loss on cardiovascular disease risk markers, Am J Clin Nutr 2009; 89: 1321-7.
24. Jan Piet Van Humburg et al., Vitamin D suppresses the pathogenic behaviour of primary Th17 cells from patients with early rheumatoid arthritis, Ann Rheum Dis 2011 70: A47.
25. Laura Dickie et al., Vitamin D3 down-regulates intracellular Toll-like receptor 9 expression and Toll-like receptor 9-induced IL-6 production in human monocytes, Rheumatology 2010; 49:1466-1471.
26. Diane Wallis et al., Contemporary Reviews in Cardiovascular Medicine—The Sunshine Deficit and Cardiovascular Disease, Circulation. 2008; 118: 1476-1485.
27. Baljit Khambha et al., Effectiveness of Vitamin D in the Treatment of Mood Disorders: A Literature Review, JOM Volume 26, Number 3, 2011 Review Article 127.
28. F. A Farraye, et al., Use of a Novel Vitamin D Bioavailability Test Demonstrates That Vitamin D Absorption is Decreased in patients with Crohn's disease, Inflamm Bowel Dis 2011; 17:2116-2121).
29. Linlin Yang, Therapeutic Effect of Vitamin D Supplementation in a Pilot Study of Crohn's Patients, Clinical and Translational Gastroenterology (2013) 4, e33; doi:10.1038/ctg.2013.1.
30. Evaluation, Treatment, and Prevention of Vitamin D deficiency—An endocrine Society Clinical Practice Guideline, Michael Holick, Journal of Clinical Endocrinology & Metabolism, July 2011, 96(7): 1911-1930.
31. Bianca, Itariu et al., Treatment with n-3 Polyunsaturated Fatty Acids Overcomes the Inverse Association of Vitamin D Deficiency with Inflammation in Severely Obese Patients: A Randomized Controlled Trial, PLoS ONE 8(1): e54634. doi:10.1371/journal.pone.0054634.
32. Prasenjit Menna, Vitamin D Up-regulates Glucose Transporter 4 (GLUT4) Translocation and Glucose Utilization, JBC, VOL. 287, NO. 50, pp. 42324-42332.
33. S. A. Masino et al., Adenosine, Ketogenic Diet and Epilepsy: The Emerging Therapeutic Relationship Between Metabolism and Brain Activity, Current Neuropharmacology, 2009, 7, 257-268 257.
34. S Boyd Eaton et al., Dietary Intake of Long-Chain Polyunsaturated Fatty Acids during the Paleolithic, World Rev Nutr Diet. Basel, Karger, 1998, vol 83, pp 12-23.
35. Manu V. Chakravarthy, et al., Eating, exercise, and “thrifty” genotypes: connecting the dots toward an evolutionary understanding of modern chronic diseases, J Appl Physiol 96: 3-10, 2004.
36. Kaori Minehira, et al., Effect of Carbohydrate Overfeeding on Whole Body and Adipose Tissue Metabolism in Humans, Obes Res. 2003; 11:1096-1103.
37. Caleb Flinch, Evolution of the human lifespan and diseases of aging: Roles of infection, inflammation, and nutrition, PNAS 2010 vol. 107, 1718-24.
38. Caleb Flinch, Evolution of the Human Lifespan, Past, Present, and Future: Phases in the Evolution of Human Life Expectancy in Relation to the Inflammatory Load, PNAS, Vol. 156, No. 1, 9-44s.
39. Lauren Constantini et al., Hypometabolism as a therapeutic target in Alzheimer's disease, BMC Neuroscience 2008, 9(Suppl 2):S16, 1-9.

1. A grain free solid-in-oil dispersion comprising at least one medium chain fatty acid (MCFA), at least one polyunsaturated fatty acid (PUFA), at least one solid,
wherein the dispersion is a grain free food product hat provides health benefits.

2. The solid-in-oil dispersion of claim 1, wherein the medium chain fatty acid comprises from about 35% to about 90% by weight of total fat in the dispersion, and the polyunsaturated fatty acid (PUFA) comprises from about 10% to about 65% of by weight of total fat in the dispersion, or acceptable salts thereof;
wherein the solid is at least about 50% by weight of the dispersion composition and is an insoluble drug, an isolated protein, a flour, or acceptable salts thereof; and
optionally further comprising one or more of dietary carriers, preservative agents or adjuvants.

3. The solid-in-oil dispersed composition of claim 1, wherein the polyunsaturated fatty acid (PUFA) is ethyl eicosapentaenoic acid (Ethyl EPA), linolenic acid (LA), arachidonic acid (AA), docosahexaenoic acid (DHA), alpha-linolenic acid (ALA), stearadonic acid (STA), eicosatrienoic acid (ETA), docosapentaenoic acid (DPA) or a nutraceutically acceptable salt or derivative thereof.

4. The solid-in-oil dispersion of claim 1, wherein the solid is protein, flour, carrier or an insoluble active agent.

5. The solid-in-oil dispersion of claim 1, wherein the medium chain fatty acid is from a whole food such as coconut flour, palm drupe flour, or camphor drupe flour.

6. The solid-in-oil dispersion of claim 1, wherein the polyunsaturated fatty acid (PUFA) is eicosapentaenoic acid (EPA) and the solid-in-oil dispersed composition contains not more than about 10%, by weight, docosahexaenoic acid.

7. The solid-in-oil dispersion of claim 1, wherein the polyunsaturated fatty acid (PUFA) is docosahexaenoic acid and the solid-in-oil dispersed composition contains not more than about 10%, by weight, eicosapentaenoic acid (EPA).

8. The solid-in-oil dispersion of claim 1, wherein the polyunsaturated fatty acid (PUFA) is docosapentaenoic acid (DPA) and the solid-in-oil dispersed composition contains not more than about 10%, combined weight of eicosapentaenoic acid (EPA) and docosahexaenoic acid (DHA).

9. The solid-in-oil dispersed composition of claim 1, wherein the solid-in-oil dispersed composition comprises at least one adjuvant, preservative, antioxidant, thickening agent, chelating agent, antifungal agent, antibacterial agent, isotonic agent, flavoring agent, sweetening agent, anti-foaming agent, colorant, diluent, moistening agent, parietal cell activator, or any combination of thereof.

10. The solid-in-oil dispersed composition of claim 1, wherein the medium chain fatty acid (MCFA), is caprylic acid.

11. The solid-in-oil dispersed composition of claim 10, wherein the medium chain fatty acid (MCFA) comprises at least about 90%, by weight, of caprylic acid.

12. The solid-in-oil dispersed composition of claim 1, wherein the medium chain fatty acid (MCFA), has from about 35% to about 90% by weight of capric acid.

13. The solid-in-oil dispersed composition of claim 12, wherein the medium chain fatty acid (MCFA) comprises at least about 90%, by weight, of capric acid.

14. The solid-in-oil dispersed composition of claim 1, further comprising an optional dietary agent such as, for example, a vitamin, an amino acid, a hormone, an element, a nutrient, and the like.

15. A nutraceutical composition comprising a grain free solid-in-oil dispersion of claim 1, having from about 35% to about 90% a medium chain fatty acids, by weight; from about 10% to about 65% of at least one polyunsaturated fatty acid (PUFA) by weight; at least one solid; wherein the solid is at least about 50% by weight of the dispersion composition; and optionally a dietary carrier or a preservative agent; or acceptable salts or derivatives thereof.

16. The composition of claim 15, wherein the medium chain fatty acid is from a whole food such as coconut flour, palm drupe flour, or camphor drupe flour.

17. The composition of claim 15, wherein the dispersion is suitable for admixing with a food product such as a breakfast cereal snacks, drink, an ice cream, a meal, or a dessert.

18. The composition of claim 15, wherein the polyunsaturated fatty acid (PUFA) is ethyl eicosapentaenoic acid (Ethyl EPA), linolenic acid (LA), arachidonic acid (AA), docosahexaenoic acid (DHA), alpha-linolenic acid (ALA), stearadonic acid (STA), eicosatrienoic acid (ETA), docosapentaenoic acid (DPA) or a nutraceutically acceptable salt or derivative thereof.

19. The composition of claim 15, further comprising an optional dietary agent such as, for example, a vitamin, an amino acid, a hormone, an element, a nutrient, and the like.
